(19) United States
(12) Patent Application Publication
(10) Pub. No.: US 2023/0382998 A1
(43) Pub. Date: Nov. 30, 2023

(54) METHODS FOR TREATING INFLAMMATORY BOWEL DISEASES WITH a4B7 INTEGRIN ANTAGONISTS

(71) Applicant: Protagonist Therapeutics, Inc., Newark, CA (US)

(72) Inventors: Suneel Kumar GUPTA, Sunnyvale, CA (US); Nishit Bachulal Modi, Sunnyvale, CA (US)

(21) Appl. No.: 18/139,254

(22) Filed: Apr. 25, 2023

Related U.S. Application Data
(60) Provisional application No. 63/334,388, filed on Apr. 25, 2022.

(51) Int. Cl. C07K 16/28; A61K 9/00; A61P 1/04

(52) U.S. Cl. CPC C07K 16/2842; A61K 9/0053; A61P 1/04

(57) ABSTRACT

The disclosure relates to compositions comprising and methods of treating inflammatory bowel diseases by administering an engineered peptide of formula (I) or a pharmaceutically acceptable salt thereof that bind α4β7 integrin.

### RELATED APPLICATION(S)

This application claims priority to U.S. Provisional Patent Appl. No. 63/334,388, filed Apr. 25, 2022, which is incorporated herein by reference in its entirety.

### FIELD OF THE INVENTION

The disclosure relates to compositions comprising, and methods of treating inflammatory bowel diseases by administering, an engineered peptide of formula (I) or a pharmaceutically acceptable salt thereof that bind α4β7 integrin.

### BACKGROUND OF THE INVENTION

Integrins are noncovalently associated α/β heterodimeric cell surface receptors involved in numerous cellular processes ranging from cell adhesion and migration to gene regulation (Dubree, et al., Selective α4β7 Integrin Antagonist and Their Potential as Anti-inflammatory Agents, J. Med. Chem. 2002, 45, 3451-3457). Differential expression of integrins can regulate a cell's adhesive properties, allowing different leukocyte populations to be recruited to specific organs in response to different inflammatory signals. If left unchecked, the integrin-mediated adhesion process can lead to chronic inflammation and autoimmune disease.

The α4 integrins, α4β1 and α4β7, play essential roles in lymphocyte migration throughout the gastrointestinal tract. They are expressed on most leukocytes, including B and T lymphocytes, where they mediate cell adhesion via binding to their respective primary ligands, vascular cell adhesion molecule (VCAM), and mucosal addressing cell adhesion molecule 1 (MAdCAM1), respectively. The proteins differ in binding specificity in that VCAM binds both α4β1 and to a lesser extent α4β7 , while MAdCAM1 is highly specific for α4β7 . In addition to pairing with the α4 subunit, the β7 subunit also forms a heterodimeric complex with αE subunit to form α4β7 , which is primarily expressed on intraepithelial lymphocytes (IEL) in the intestine, lung, and genitourinary tract. α4β7 is also expressed on dendritic cells in the gut. The α4β7 heterodimer binds to E-cadherin on the epithelial cells. The IEL cells are thought to provide a mechanism for immune surveillance within the epithelial compartment. Therefore, blocking α4β7 and α4β7 together may be a useful method for treating inflammatory conditions of the intestine.

Inhibitors of specific integrins-ligand interactions have been shown effective as anti-inflammatory agents for the treatment of various autoimmune diseases. For example, monoclonal antibodies displaying high binding affinity for α4β7 have displayed therapeutic benefits for gastrointestinal auto-inflammatory/autoimmune diseases, such as Crohn's disease, and ulcerative colitis (Id). However, these therapies interfered with α4β1 integrin-ligand interactions, thereby resulting in dangerous side effects to the patient. Therapies utilizing small molecule antagonists have shown similar side effects in animal models, thereby preventing further development of these techniques. More recently engineered peptides showing high potency and stability, as well as high specificity for α4β7 integrins, have been shown to be effective in the treatment of various immune disorders, including inflammatory bowel disease.

However, there is a need in the art for additional methods of using α4β7 antagonists and other agents for treating inflammatory disorders. The present application meets this and other needs.

### SUMMARY OF THE INVENTION

The present disclosure provides composition and methods for treating various diseases and conditions associated with α4!37 integrin signaling and methods of optimizing or improving therapeutic efficacy.

In one aspect, the disclosure provides a method of treating an inflammatory bowel disease (IBD) in a subject in need thereof, the method comprising orally administering to the subject a compound of formula (I):

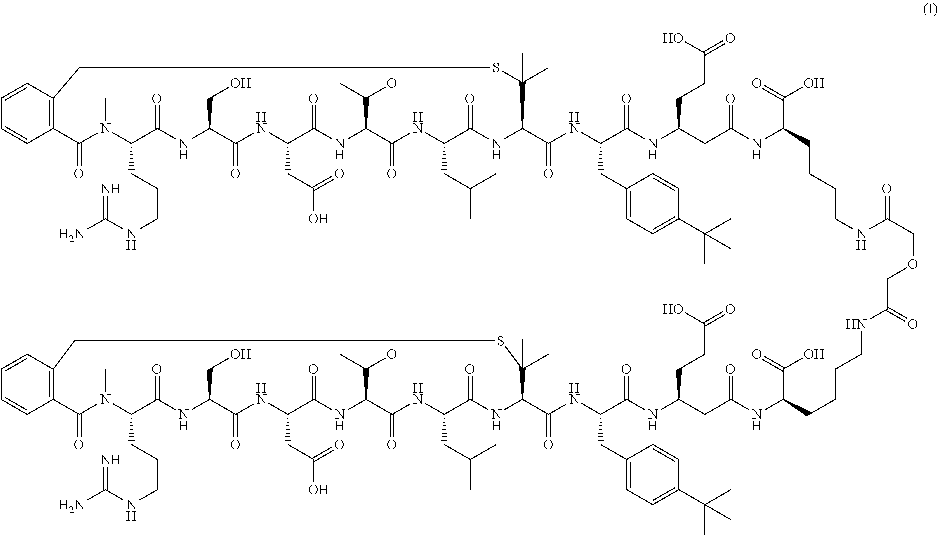 [SMILES: CC(C)C[C@@H]1NC(=O)[C@H](C(C)O)NC(=O)[C@H](CC(=O)O)NC(=O)[C@H](CO)NC(=O)[C@H](CCCNC(=N)N)N(C)C(=O)c2ccccc2CSC(C)(C)[C@@H](C(=O)N[C@@H](Cc2ccc(C(C)(C)C)cc2)C(=O)N[C@@H](CCC(=O)O)CC(=O)N[C@H](CCCCNC(=O)COCC(=O)NCCCC[C@@H](NC(=O)C[C@H](CCC(=O)O)NC(=O)[C@H](Cc2ccc(C(C)(C)C)cc2)NC(=O)[C@H]2NC(=O)[C@H](CC(C)C)NC(=O)[C@H](C(C)O)NC(=O)[C@H](CC(=O)O)NC(=O)[C@H](CO)NC(=O)[C@H](CCCNC(=N)N)N(C)C(=O)c3ccccc3CSC2(C)C)C(=O)O)C(=O)O)NC1=O]

or a pharmaceutically acceptable salt thereof, or a pharmaceutical composition comprising a compound of formula (1) or a pharmaceutically acceptable salt thereof, wherein the subject has a plasma concentration of the compound at from about 0.1 ng/mL to about 4 ng/mL and wherein the administering compound of formula (I) to the subject achieves a clinical remission rate of at least about 6% higher relative to a clinical remission rate from placebo.

In another aspect, the disclosure provides a method of optimizing or improving therapeutic efficacy for treating IBD in a subject in need thereof, the method comprising orally administering a compound of formula (I):

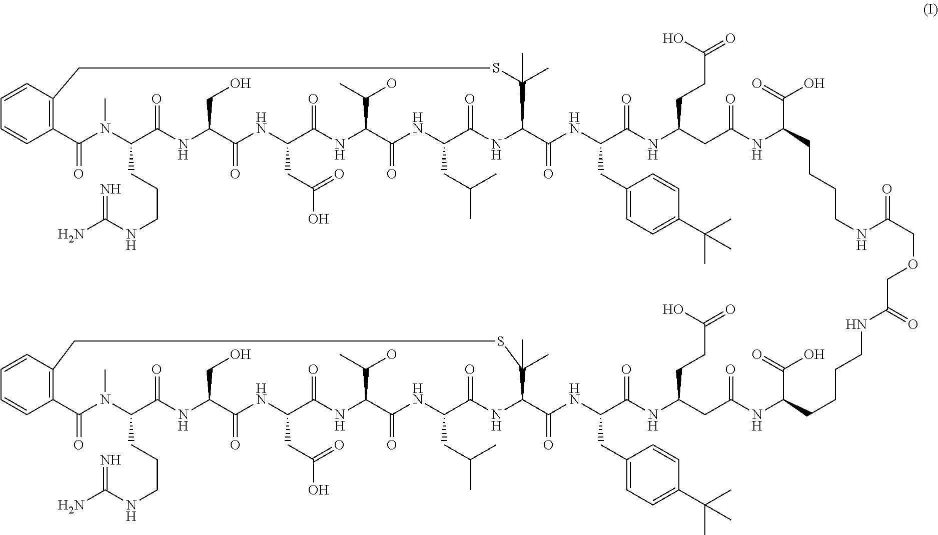 [SMILES: CC(C)C[C@@H]1NC(=O)[C@H](C(C)O)NC(=O)[C@H](CC(=O)O)NC(=O)[C@H](CO)NC(=O)[C@H](CCCNC(=N)N)N(C)C(=O)c2ccccc2CSC(C)(C)[C@@H](C(=O)N[C@@H](Cc2ccc(C(C)(C)C)cc2)C(=O)N[C@@H](CCC(=O)O)CC(=O)N[C@H](CCCCNC(=O)COCC(=O)NCCCC[C@@H](NC(=O)C[C@H](CCC(=O)O)NC(=O)[C@H](Cc2ccc(C(C)(C)C)cc2)NC(=O)[C@H]2NC(=O)[C@H](CC(C)C)NC(=O)[C@H](C(C)O)NC(=O)[C@H](CC(=O)O)NC(=O)[C@H](CO)NC(=O)[C@H](CCCNC(=N)N)N(C)C(=O)c3ccccc3CSC2(C)C)C(=O)O)C(=O)O)NC1=O]

or a pharmaceutically acceptable salt thereof, or a pharmaceutical composition comprising a compound of formula (I) or a pharmaceutically acceptable salt thereof, to the subject, wherein the plasma concentration of the compound of formula (I) in the subject is maintained at a predetermined concentration. In particular embodiments, the plasma concentration is maintained at the predetermined concentration over the course of treatment. In particular embodiments, the plasma concentration is maintained at the predetermined concentration over the majority of the course of treatment.

In another embodiment, the methods include administering a compound of formula (I) or pharmaceutically acceptable salt thereof. In certain embodiments, the compound of formula (I) is a hydrochloride salt. In another embodiment, the compound of formula (I) is an acetate salt.

In another embodiment, the present invention provides pharmaceutical compositions comprising the compound of formula (I) or pharmaceutically acceptable salt thereof, and a pharmaceutically acceptable excipient.

In another embodiment, the pharmaceutical composition is formulated for a route of administration selected from oral administration, parenteral administration, buccal administration, nasal administration, topical administration, and rectal administration. In particular embodiments, the pharmaceutical composition is formulated for oral administration.

In another embodiment, the invention includes a pharmaceutical composition comprising a compound of formula (I) or pharmaceutically acceptable salt thereof disclosed herein and a pharmaceutically acceptable carrier, diluent or excipient. In particular embodiments, the pharmaceutical composition is formulated for oral delivery. In certain embodiments, it further comprises an enteric coating. In certain embodiments, the enteric or delayed release coating releases the pharmaceutical composition within a subject's lower gastrointestinal system.

In a further embodiment, the invention provides a method for treating or preventing a disease or condition that is associated with a biological function of integrin α4β7 , the method comprising providing to a subject in need thereof an effective amount of a compound of formula (I) or pharmaceutically acceptable salt thereof, or a pharmaceutical composition of the invention. In certain embodiments, the disease or condition is an inflammatory bowel disease. In particular embodiments, the inflammatory bowel disease is ulcerative colitis (e.g., moderate to severe ulcerative colitis) or Crohn's disease. In particular embodiments, the compound of formula (I) or pharmaceutically acceptable salt thereof inhibits binding of α4β7 to MAdCAM. In certain embodiments, the compound of formula (I) or pharmaceutically acceptable salt thereof or the pharmaceutical composition is provided to the subject in need thereof at an interval sufficient to ameliorate the condition. In certain embodiments, the interval is selected from the group consisting of around the clock, hourly, every four hours, once daily, twice daily, three times daily, four times daily, every other day, weekly, bi-weekly, and monthly. In particular embodiments, the compound of formula (I) or pharmaceutically acceptable salt thereof or pharmaceutical composition is provided as an initial dose followed by one or more subsequent doses, and the minimum interval between any two doses is a period of less than 1 day, and wherein each of the doses comprises an effective amount of the compound of formula (I) or pharmaceutically acceptable salt thereof. In particular embodiments, the compound or pharmaceutically acceptable sat thereof is administered once daily or twice daily. In particular embodiments, the effective amount of the compound of formula (I) or pharmaceutically acceptable salt thereof or the pharmaceutical composition is sufficient to achieve at least one of the following: a) about 50% or greater saturation of MAdCAM binding sites on α4β7 integrin molecules; b) about 50% or greater inhibition of α4β7 integrin expression on the cell surface; and c) about 50% or greater saturation of MAdCAM binding sites on α4β7 molecules and about 50% or greater inhibition of α4β7 integrin expression on the cell surface, wherein i) the saturation is maintained for a period consistent with a dosing frequency of no more than twice daily; ii) the inhibition is maintained for a period consistent with a dosing frequency of no more than twice daily; or iii) the saturation and the inhibition are each maintained for a period consistent with a dosing frequency of no more than twice daily. In certain embodiments, the peptide molecule is administered orally, parenterally, or topically.

In some embodiments, a compound of formula (I) or pharmaceutically acceptable salt thereof or a pharmaceutical composition provided herein is administered to a human.

In some embodiments, compositions provided herein are orally administered.

In certain embodiments, the invention encompasses a method of treating an inflammatory bowel disease (IBD) in a subject in need thereof, the method comprising orally administering to the subject a compound of formula (I): or a pharmaceutically acceptable salt thereof, or a pharmaceutical composition comprising a compound of formula (I) or a pharmaceutically acceptable salt thereof, wherein the subject has a plasma concentration of the compound at from about 0.1 ng/mL to about 4 ng/mL and wherein the administering compound of formula (I) to the subject achieves a clinical remission rate of at least about 6% higher relative to a clinical remission rate from placebo.

In certain embodiments, the plasma concentration is between about 0.2 and about 0.7 ng/mL.

In certain embodiments, the compound is administered at a dose from about 50 mg to about 1000 mg per day as divided doses or as a once daily dose.

In certain embodiments, the compound is administered to the subject at a dose of about 62.5, 75, 87.5, 100.0, 112.5, 125.0, 137.5, 150.0, 162.5, 175, 187.5, 200.0, 212.5, 225.0, 237.5, 250.0, 262.5, 275, 287.5, 300.0, 312.5, 325.0, 337.5, 350.0, 362.5, 375, 387.5, 400.0, 412.5, 425.0, 437.5, 450.0, 462.5, 475, 487.5, 500.0, 512.5, 525, 537.5, 550, 562.5, 575, 587.5, 600, 612.5, 625, 637.5, 650, 662.5, 675, 687.5, 700, 712.5, 725, 737.5, 750, 762.5, 775, 787.5,800, 812.5, 825, 837.5, 850, 862.5, 875, 887.5, 900, 912.5, 925, 937.5, 950, 962.5, 975, 987.5, 1000, 1112.5, 1125.0, 1137.5, 1150.0, 1162.5, 1175, 1187.5, 1200.0, 1212.5, 1225.0, 1237.5, 1250.0, 1262.5,1275, 1287.5, 1300.0, 1312.5, 1325.0, 1337.5, 1350.0, 1362.5, 1375, 1387.5, or 1400.0 mg.

In certain embodiments, the compound is administered at a dose of 150 mg or 450 mg once or twice per day.

In certain embodiments, the administration of a compound of formula (I) to the subject achieves the clinical remission rate from about 13% to about 40% higher relative to the clinical remission rate from placebo.

In certain embodiments, the administering compound of formula (I) to the subject achieves the clinical remission rate of at least 40% higher relative to the clinical remission rate from placebo.

In certain embodiments, the administering compound of formula (I) to the subject achieves a histologic remission rate of at least about 20% higher relative to the histologic remission rate from placebo.

In certain embodiments, the administering compound of formula (I) to the subject achieves a histologic remission rate of at least about 20% to about 37% higher relative to the histologic remission rate from placebo.

In certain embodiments, the invention encompasses a method of optimizing or improving therapeutic efficacy for treating IBD in a subject in need thereof, the method comprising orally administering a compound of formula (I) or a pharmaceutically acceptable salt thereof, or a pharmaceutical composition comprising a compound of formula (I) or a pharmaceutically acceptable salt thereof to the subject, wherein the plasma concentration of the compound of formula (I) in the subject is maintained at a predetermined concentration.

In certain embodiments, the predetermined plasma concentration is between about 0.1 and about 0.85 ng/mL.

In certain embodiments, the predetermined plasma concentration is between about 0.3 and about 0.7

In certain embodiments, the plasma concentration is maintained by determining the plasma concentration of the compound of formula (I) in the subject; and adjusting administering the compound of formula (I) or a pharmaceutically acceptable salt thereof or a pharmaceutical composition comprising a compound of formula (I) or a pharmaceutically acceptable salt thereof to achieve a desired therapeutic effect.

In certain embodiments, the adjusting comprises changing the dosage regimen of the compound administered to the subject.

In certain embodiments, the compound of formula (I) or a pharmaceutically acceptable salt thereof is administered twice daily.

In certain embodiments, the subject is a human patient.

In certain embodiments, the IBD is ulcerative colitis.

In certain embodiments, the subject has moderate to severe ulcerative colitis.

In certain embodiments, the IBD is Crohn's disease.

Articles of manufacture including packaging material, a compound of formula (I) or pharmaceutically acceptable salt thereof or composition or pharmaceutically acceptable derivative thereof provided herein, which is effective for treating IBD or related conditions, within the packaging material, and a label that indicates that the compound or composition, or pharmaceutically acceptable salt, pharmaceutically active metabolite, pharmaceutically acceptable prodrug, or pharmaceutically acceptable solvate thereof, is used for treating the IBD or related conditions, are provided.

In a related aspect, the disclosure provides a pharmaceutical composition comprising a compound of formula (I) or a pharmaceutically acceptable salt thereof. In particular embodiments, the composition is formulated for oral delivery, optionally wherein the composition comprises an enteric coating. In particular embodiments, the method comprises administering to the subject the pharmaceutical composition disclosed herein.

In particular embodiments of methods and compositions disclosed herein, the antagonist or pharmaceutically acceptable salt thereof inhibits binding of α4β7 integrin to MAdCAM1.

In particular embodiments of methods and compositions disclosed herein, the compound of formula (I) or a pharmaceutically acceptable salt thereof or the pharmaceutical composition is provided to the subject in need thereof at an interval sufficient to improve or ameliorate the condition. In particular embodiments, the interval is selected from the group consisting of: around the clock, hourly, every four hours, once daily, twice daily, three times daily, four times daily, every other day, weekly, bi-weekly, and monthly. In certain embodiments, the compound of formula (I) or a pharmaceutically acceptable salt thereof or pharmaceutical composition is provided as an initial does followed by one or more subsequent doses, and the minimum interval between any two doses is a period of less than 1 day, and wherein each of the doses comprises an effective amount of the compound of formula (I) or a pharmaceutically acceptable salt thereof. In some embodiments, the effective amount of the compound of formula (I) or a pharmaceutically acceptable salt thereof or the pharmaceutical composition is sufficient to achieve at least one of the following.
a) about 50% or greater saturation of MAdCAM1 binding sites on α4β7 integrin molecules;
b) about 50% or greater inhibition of α4β7 integrin expression on the cell surface; or
c) about 50% or greater saturation of MAdCAM1 binding sites on α4β!7 molecules and about 50% or greater inhibition of α4β7 integrin expression on the cell surface, wherein i) the saturation is maintained for a period consistent with a dosing frequency of no more than twice daily; ii) the inhibition is maintained for a period consistent with a dosing frequency of no more than twice daily; or iii) the saturation and the inhibition are each maintained for a period consistent with a dosing frequency of no more than twice daily.

Other objects, features and advantages of the methods and compositions described herein will become apparent from the following detailed description. It should be understood, however, that the detailed description and the specific examples, while indicating specific embodiments, are given by way of illustration only, since various changes and modifications within the spirit and scope of the present disclosure will become apparent to those skilled in the art from this detailed description. The section headings used herein are for organizational purposes only and are not to be construed as limiting the subject matter described. All documents, or portions of documents, cited in the application including, but not limited to, patents, patent applications, articles, books, manuals, and treatises are hereby expressly incorporated by reference in their entirety for any purpose.

### BRIEF DESCRIPTION OF THE DRAWINGS

FIGS. 1A and 1B illustrate the effect of the compound of formula (I) dose on clinical remission in all subjects (FIG. 1A) and bionaive subjects (FIG. 1B). Modified Intent to Treat (mITT) analysis.

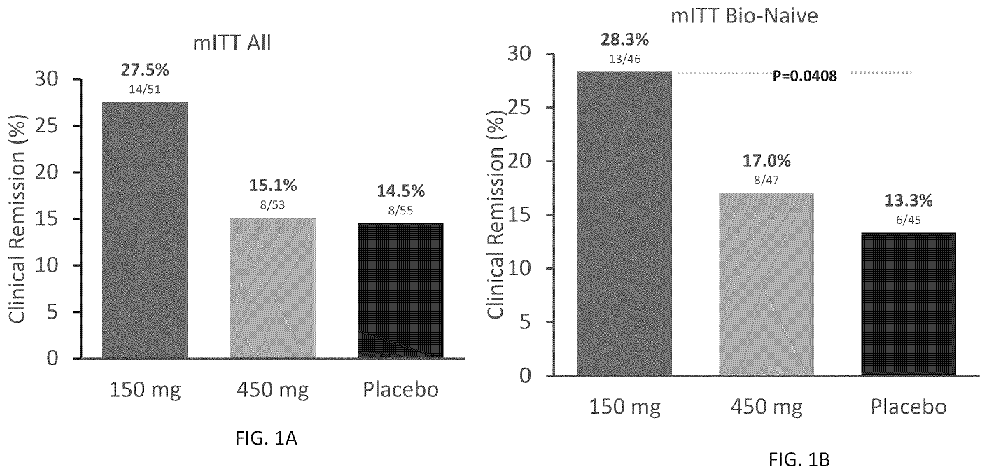

FIG. 2 illustrates the compound of formula (I) trough concentration quartiles at week 12 relative to proportion of subjects achieving clinical remission at Week 12.

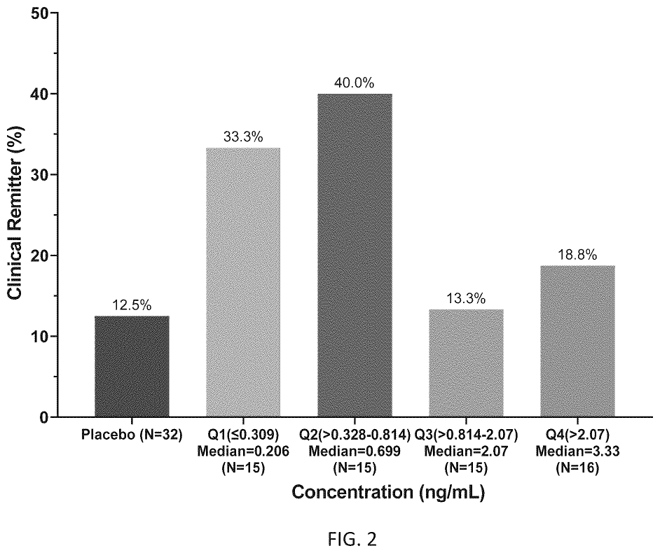

FIG. 3 illustrates the compound of formula (I) trough concentration quartiles at week 12 segmented by compound (I) dose relative to proportion of subjects achieving clinical remission at Week 12. For each bar, the lighter shade represents 150 mg BID and darker share represents 450 mg BID dose. Thus, for Q1, the total is 33.3%, with 26.7% being 450 mg BID dose; for Q2, the total is 40%, with 26.7% being 450 mg BID dose; for Q3, the total is 13.3%, with 6.7% being 450 mg BID dose; and for Q4, the total is 18.8%, with 6.3% being 450 mg BID dose.

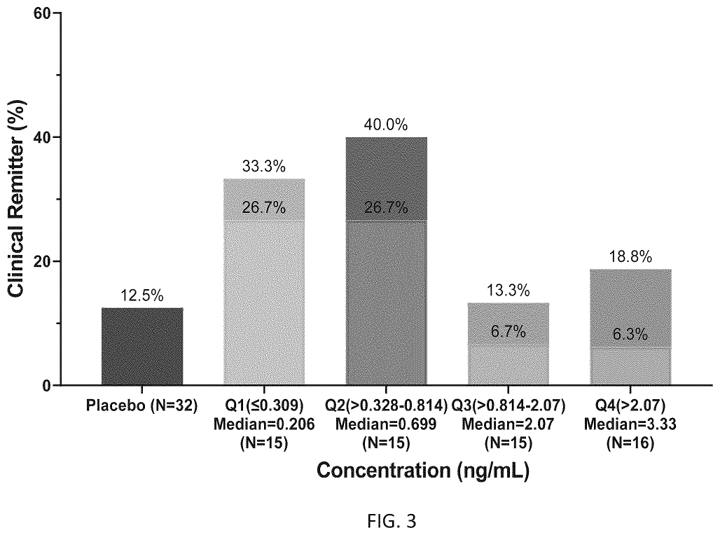

FIGS. 4A and 4B illustrate the effect of the compound of formula (I) dose on histologic remission (FIG. 4A) and histologic improvement (FIG. 4B). RHI=Robarts Histological Index.

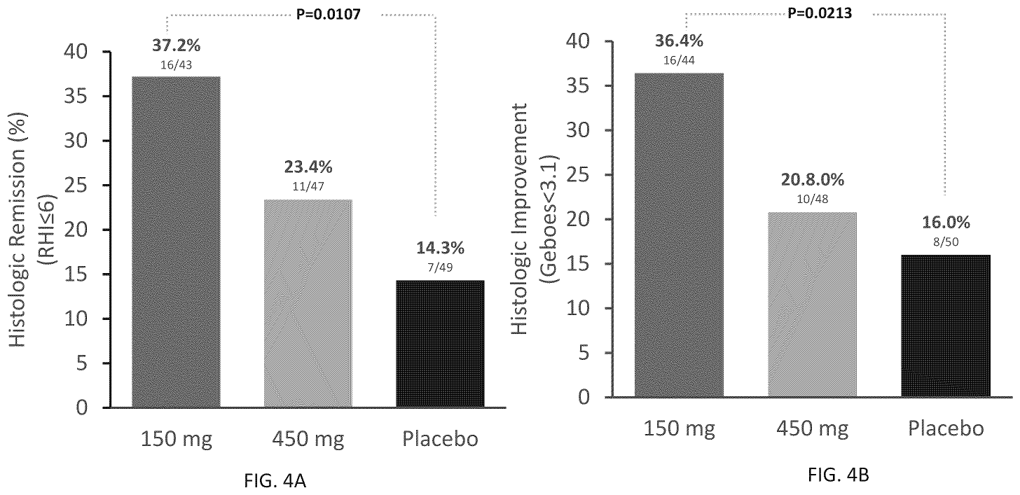

### DETAILED DESCRIPTION OF THE INVENTION

Ulcerative colitis is a chronic inflammatory bowel disease (IBD) with a remitting and relapsing course, characterized by bloody diarrhea, abdominal cramps, and fatigue. The pathogenesis is thought to result from inappropriate immune response to gastrointestinal antigens and environmental triggers in genetically susceptible individuals. The highest prevalence is reported in Europe and North America. Ulcerative colitis has a significant negative impact on patient quality of life and presents a high economic burden on health systems.

Inflammatory bowel diseases, such as ulcerative colitis, have been managed with corticosteroids, 5-aminosalicylates, and immunosuppressants, and more recently, with the use of biologics targeted against specific mediators of inflammation. Therapeutic options for the long-term treatment of ulcerative colitis are limited. 5-aminosalicylates such as sulfasalazine, olsalazine, balsalazide and various forms of mesalamine (e.g., Asacol, Pentasa, Lialda, Canasa) are only effective in mild- to moderate disease whereas patients with severe disease may be started on biologics. Several monoclonal antibodies against TNF-α (e.g., infliximab, adalimumab, golimumab, and certolizumab) are now available. Agents targeted against other cytokines involved in the inflammatory response such as ustekinumab against IL-12/IL-23, and tofacitinib, a pan-JAK inhibitor, are now part of the therapeutic options available for inflammatory bowel disease, and several IL-23 and S1P1 inhibitors are also currently under clinical investigation.

In spite of the wide array of therapeutic options, there are still limitations in the treatment of inflammatory bowel diseases and the agents available are not without risk. TNF-α inhibitors are ineffective in approximately ⅕ to ⅓ of the patients and 10-15% of treated patients who show an initial benefit may lose response every year. Cutaneous reactions are also the most common adverse reactions with anti-TNF treatments. This includes injection site reactions, cutaneous infections, immune-mediated complications such as psoriasis and lupus-like syndrome and rarely skin cancers. Tofacitinib can increase the risk of infection and may increase the risk of thrombosis or thromboembolic events. There is increasing recognition that mitigation of the local inflammatory response may hold promise. Orally administered budesonide and 5-ASAs are effective locally, and various other locally acting agents, including AMT-101, a locally acting oral biologic fusion protein of interleukin 10, and TD-1473, a JAK inhibitor, have shown promise or are undergoing clinical investigation. Local delivery through oral administration may allow higher doses of drug to be delivered to the target site without increasing systemic side effects.

Integrins are heterodimers that function as cell adhesion molecules. The α4 integrins, α4β1 and α4β7 , are known to play essential roles in lymphocyte migration throughout the gastrointestinal tract. They are expressed on most leukocytes, including B and T lymphocytes, monocytes, and dendritic cells, where they mediate cell adhesion via binding to their respective primary ligands, namely vascular cell adhesion molecule (VCAM) and mucosal addressin cell adhesion molecule 1 (MAdCAM1). VCAM and MAdCAM1 differ in binding specificity, in that VCAM binds both α4β1 and α4β7 , while MAdCAM1 is highly specific for α4β7.

The α4β7 integrin, which is primarily involved in the recruitment of leukocytes to the gastrointestinal (GI) tract, is present on the cell surface of a small population of circulating T and B lymphocytes. Its major ligand, MAdCAM1 is selectively expressed on the endothelium of the intestinal vasculature and is present in increased concentrations in inflamed tissue.

The present disclosure provides methods of treating IBDs by inhibiting α4β7 integrin, for example, using a compound of formula (I) or a pharmaceutically acceptable salt thereof. In particular, the disclosure provides oral dosages of α4β7 integrin antagonists effective in treating IBDs, including ulcerative colitis. In addition, the disclosure provides pharmacokinetic and pharmacodynamics parameters of α4β7 integrin antagonists associated with biological activity of the antagonists, such as inhibition of MAdCAM1-mediated T cell proliferation, reduced T cell expression of β7 (and α4β7 integrin), internalization of α4β7 integrin on T cells, reduced homing of T cells into gastrointestinal tract tissue, decreased cytokine release by T cells, reduced adhesion of T cells to MAdCAM1, and reduced gastrointestinal tract inflammation. In particular embodiments, the T cells are CD4+ T memory cells.

Furthermore, it was previously believed that the mechanism underlying the use of α4β7 integrin antagonists to treat IBDs involved binding of the antagonist to α4β7 expressed on circulating T cells, which prevents the T cells from binding to MAdCAM1 expressed on GI endothelial cells, thus preventing extravascular migration of the T cells into the inflamed gastrointestinal mucosa of IBD patients. Thus, a goal was to achieve maximum blood receptor occupancy (% RO), e.g., greater than 80% RO, greater than 90% RO, or close to 100% RO, in order to block binding and migration of the T cells into the inflamed gastrointestinal mucosa.

The present inventors have identified an alternative mechanism by which α4β7 integrin antagonists inhibit inflammation within inflamed tissue, such as inflamed gastrointestinal mucosa, by exerting a local effect, which was reported in PCT Application Publication No. WO 2021/142,373, the disclosure of which is incorporated herein by reference in its entirety. α4β7-Integrin antagonist, when present in the inflamed tissue, are able to inhibit MAdCAM1-mediated CD4+ T cell proliferation and cytokine production that occurs through direct binding and stimulation of α4β7 integrin. This local effect does not require blood receptor occupancy saturation, but instead, oral administration of a sub-saturating dose of the antagonist is sufficient to achieve a therapeutic effect, e.g., endoscopic improvement or histological improvement. Thus, the disclosure provides, inter alia, methods of treating IBDs that comprise orally providing to a subject a sub-saturating blood receptor occupancy amount of an α4β7 integrin antagonist, including but not limited to the compound of formula (I) or a pharmaceutically acceptable salt thereof.

In certain aspects, the present disclosure provides methods of using a compound of formula (I) or a pharmaceutically acceptable salt thereof as anti-inflammatory and/or immunosuppressive agents, e.g., for use in treating a condition that is associated with a biological function of α4β7 or on cells or tissues expressing MAdCAM1.

Aspects of the invention relate to a compound of formula (I) or a pharmaceutically acceptable salt thereof exhibiting integrin antagonist activity, namely, exhibiting high specificity for α4β7 integrin. In certain embodiments, the compound of formula (I) or a pharmaceutically acceptable salt thereof demonstrate increased stability when administered orally as a therapeutic agent.

In a further related embodiment, the present invention provides a method for treating or preventing a disease or condition that is associated with a biological function of integrin α4β7, the method comprising providing to a subject in need thereof an effective amount of a compound of formula (I) or a pharmaceutically acceptable salt thereof or a pharmaceutical composition of the invention. In certain embodiments, the disease or condition is an inflammatory bowel disease. In particular embodiments, the inflammatory bowel disease is ulcerative colitis (e.g., moderate to severe acute ulcerative colitis) or Crohn's disease. In particular embodiments, the compound of formula (I) or a pharmaceutically acceptable salt thereof inhibits binding of α4β7 to MAdCAM1. In certain embodiments, the compound of formula (I) or a pharmaceutically acceptable salt thereof or the pharmaceutical composition is provided to the subject in need thereof at an interval sufficient to ameliorate the condition. In certain embodiments, the interval is selected from the group consisting of around the clock, hourly, every four hours, once daily, twice daily, three times daily, four times daily, every other day, weekly, bi-weekly, and monthly.

In particular embodiments, the compound of formula (I) or a pharmaceutically acceptable salt thereof or pharmaceutical composition is provided as an initial dose followed by one or more subsequent doses, and the minimum interval between any two doses is a period of less than 1 day, and wherein each of the doses comprises an effective amount of the compound of formula (I) or a pharmaceutically acceptable salt thereof. In particular embodiments, the effective amount of the compound of formula (I) or a pharmaceutically acceptable salt thereof or the pharmaceutical composition is sufficient to achieve at least one of the following: a) about 50% or greater saturation of MAdCAM1 binding sites on α4β7 integrin molecules; b) about 50% or greater inhibition of α4β7 integrin expression on the cell surface; and c) about 50% or greater saturation of MAdCAM1 binding sites on α4β7 molecules and about 50% or greater inhibition of α4β7 integrin expression on the cell surface, wherein i) the saturation is maintained for a period consistent with a dosing frequency of no more than twice daily; ii) the inhibition is maintained for a period consistent with a dosing frequency of no more than twice daily; or iii) the saturation and the inhibition are each maintained for a period consistent with a dosing frequency of no more than twice daily. In certain embodiments, the peptide molecule is administered orally, parenterally, or topically.

The α4β7 integrin, which is primarily involved in the recruitment of leukocytes to the gastrointestinal (GI) tract, is present on the cell surface of a small population of circulating T and B lymphocytes. Its major ligand, MAdCAM1 is selectively expressed on the endothelium of the intestinal vasculature and is present in increased concentrations in inflamed tissue.

### Definitions

As used herein, the singular forms “a,” “and” and “the” include plural references unless the context clearly dictates otherwise.

As used herein, “about” means to include (and describes) embodiments that are directed to that value or parameter per se. In certain embodiments, the term “about” or “approximate” or “approximately” includes the indicated amount ±10%. In other embodiments, the term “about” approximate” or “approximately” includes the indicated amount ±5%. In certain other embodiments, the term “about” approximate” or “approximately” includes the indicated amount ±1%.

The term “acylating organic compounds,” as used herein refers to various compounds with carboxylic acid functionality, which may be used to acylate the C- and/or N-termini of a compound of formula (I) or pharmaceutically acceptable salt thereof Non-limiting examples of acylating organic compounds include cyclopropylacetic acid, 4-Fluorobenzoic acid, 4-fluorophenylacetic acid, 3-Phenylpropionic acid, Succinic acid, Glutaric acid, Cyclopentane carboxylic acid, glutaric acid, succinic acid, 3,3,3-trifluoropropeonic acid, 3-Fluoromethylbutyric acid.

Generally, the names of naturally occurring and non-naturally occurring aminoacyl residues used herein follow the naming conventions suggested by the IUPAC Commission on the Nomenclature of Organic Chemistry and the IUPAC-IUB Commission on Biochemical Nomenclature as set out in “Nomenclature of α-Amino Acids (Recommendations, 1974)” Biochemistry, 14(2), (1975). To the extent that the names and abbreviations of amino acids and aminoacyl residues employed in this specification and appended claims differ from those suggestions, they will be made clear to the reader.

All peptide sequences are written according to the generally accepted convention whereby the α-N-terminal amino acid residue is on the left and the α-C-terminal is on the right. As used herein, the term “α-N-terminal” refers to the free α-amino group of an amino acid in a peptide, and the term “α-C-terminal” refers to the free α-carboxylic acid terminus of an amino acid in a peptide. The term “amino acid” or “any amino acid” as used here refers to any and all amino acids, including naturally occurring amino acids (e.g., a-amino acids), unnatural amino acids, modified amino acids, and non-natural amino acids. It includes both D- and L-amino acids. Natural amino acids include those found in nature, such as, e.g., the 23 amino acids that combine into peptide chains to form the building-blocks of a vast array of proteins. These are primarily L stereoisomers, although a few D-amino acids occur in bacterial envelopes and some antibiotics. The “non-standard,” natural amino acids are pyrrolysine (found in methanogenic organisms and other eukaryotes), selenocysteine (present in many noneukaryotes as well as most eukaryotes), and N-formylmethionine (encoded by the start codon AUG in bacteria, mitochondria and chloroplasts). “Unnatural” or “non-natural” amino acids are non-proteinogenic amino acids (i.e., those not naturally encoded or found in the genetic code) that either occur naturally or are chemically synthesized. Over 140 natural amino acids are known and thousands of more combinations are possible. Examples of “unnatural” amino acids include β-amino acids (β³ and β²), homo-amino acids, proline and pyruvic acid derivatives, 3-substituted alanine derivatives, glycine derivatives, ring-substituted phenylalanine and tyrosine derivatives, linear core amino acids, diamino acids, D-amino acids, alpha-methyl amino acids and N-methyl amino acids. Unnatural or non-natural amino acids also include modified amino acids. “Modified” amino acids include amino acids (e.g., natural amino acids) that have been chemically modified to include a group, groups, or chemical moiety not naturally present on the amino acid.

The term “bio-naïve” refers to two specific categories: 1) patients never previously exposed to a compound of formula (I) (primary naive), and 2) patients with previous exposure to the compound of formula (I) after an adequate washout period (secondary naive).

When the term “comprising” is used herein, it is understood that the present invention also includes the same embodiments wherein the term “comprising” is substituted with “consisting essentially of” or “consisting of”.

As used in the present specification the following terms have the meanings indicated:

The term “carboxy,” as used herein, refers to —CO₂H.

As used herein, the term “clinical remission rate” refers to when the symptoms of IBD have lessened to the point that they're mostly absent or gone, including endoscopic remission, wherein inflammation is seen during a colonoscopy or a sigmoidoscopy and determined by a healthcare provider that the lining of the digestive tract is improved or healed. As used herein, clinical remission is determined using the Adapted Mayo score (sum of 3 subscores from the Mayo score) of stool frequency, rectal bleeding, and endoscopy:
Stool frequency subscore (SFS)≤1, and
Rectal bleeding subscore (RBS) of 0, and
Endoscopic subscore (ESS)≤1 (modified so that a score of 1 does not include friability). As used herein, the term “compound of formula (I)” or Compound (I) refers to a peptide compound having the following structure:

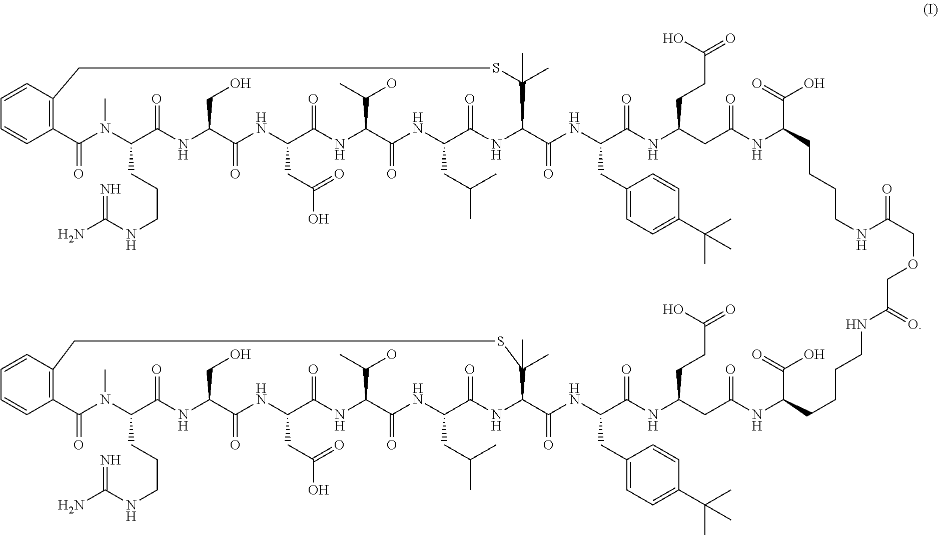 [SMILES: CC(C)C[C@@H]1NC(=O)[C@H](C(C)O)NC(=O)[C@H](CC(=O)O)NC(=O)[C@H](CO)NC(=O)[C@H](CCCNC(=N)N)N(C)C(=O)c2ccccc2CSC(C)(C)[C@@H](C(=O)N[C@@H](Cc2ccc(C(C)(C)C)cc2)C(=O)N[C@@H](CCC(=O)O)CC(=O)N[C@H](CCCCNC(=O)COCC(=O)NCCCC[C@@H](NC(=O)C[C@H](CCC(=O)O)NC(=O)[C@H](Cc2ccc(C(C)(C)C)cc2)NC(=O)[C@H]2NC(=O)[C@H](CC(C)C)NC(=O)[C@H](C(C)O)NC(=O)[C@H](CC(=O)O)NC(=O)[C@H](CO)NC(=O)[C@H](CCCNC(=N)N)N(C)C(=O)c3ccccc3CSC2(C)C)C(=O)O)C(=O)O)NC1=O]

The chemical structure of the peptide compound of formula (I) is shown above. The peptide compound of formula (I) consists of two peptide fragments connected by a diglycolic acid (DIG) linker through the ε-amino groups on the side chains of D-lysine residues at the c-terminals of the peptide fragments, wherein each peptide fragment has a sequence of amino acids represented by (2-Benzyl)-(N-Me-R)-Ser-Asp-Thr-Leu-Pen-(Phe(4-tBu))-(β-homo-Glu)-(D-Lys)-OH (SEQ ID NO:1), wherein a thioether bond is formed between the methyl group of 2-methylbenzoyl and the thio group of the Pen residue in each peptide fragment. The peptide compound of formula (I) can also be represented by (2-Benzyl)-(N-Me-R)-Ser-Asp-Thr-Leu-Pen-(Phe(4-tBu))-((3-homo-Glu)-(D-Lys)-DIG-(D-Lys)-((3-homo-Glu)-(Phe(4-tBu))-Pen-Leu-Thr-Asp-Ser-(N-Me-R)-(2-Benzyl), wherein the methyl group of the 2-methylbenzoyl moiety and the thio group of the Pen residue in each peptide fragment are taken together to form a thioether bond.

The term “cyclized,” as used herein, refers to a reaction in which one part of a compound of formula (I) or pharmaceutically acceptable salt thereof becomes linked to another part of the polypeptide molecule to form a closed ring, such as by forming a disulfide or thioether bond. In particular embodiments, peptide monomers and monomer subunits of peptide dimers of the present invention are cyclized via an intramolecular disulfide or thioether bond.

The term “dimer,” as used herein, refers broadly to a peptide comprising two monomer peptide subunits (e.g., thioether monomer peptides) that are linked at their respective C- or N-terminuses. Dimers of the present invention may include homodimers or heterodimers that function as integrin antagonists. The term “dimer” may also be referred to herein to as a “peptide dimer,” “peptide dimer molecule,” “dimer peptide,” or “dimer compound.” The term “monomer peptide subunit” may also be referred to herein as “monomer subunit,” “peptide monomer subunit,” “peptide subunit,” “peptide dimer subunit,” “dimer subunit,” “monomeric subunit,” or “subunit of a peptide dimer.”

The term “integrin-related diseases,” as used herein, refer to indications that manifest as a result of integrin binding, and which may be treated through the administration of an integrin antagonist.

The term “isotere” or “isostere replacement,” as used herein, refers to any amino acid or other analog moiety having chemical and/or structural properties similar to a specified amino acid. In particular embodiments, an “isostere” or “suitable isostere” of an amino acid is another amino acid of the same class, wherein amino acids belong to the following classes based on the propensity of the side chain to be in contact with polar solvent like water: hydrophobic (low propensity to be in contact with water), polar or charged (energetically favorable contact with water). The charged amino acid residues include lysine (+), arginine (+), aspartate (−) and glutamate (−). Polar amino acids include serine, threonine, asparagine, glutamine, histidine and tyrosine. The hydrophobic amino acids include alanine, valine, leucine, isoleucine, proline, phenylalanine, tryptophane, cysteine and methionine. The amino acid glycine does not have a side chain and is hard to assign to one of the above classes. However, glycine is often found at the surface of proteins, often within loops, providing high flexibility to these regions, and an isostere may have a similar feature. Proline has the opposite effect, providing rigidity to the protein structure by imposing certain torsion angles on the segment of the polypeptide chain.

The term “linker,” as used herein, refers broadly to a chemical structure that is capable of linking together two thioether monomer subunits to form a dimer.

The term “L-amino acid,” as used herein, refers to the “L” isomeric form of a peptide, and conversely the term “D-amino acid” refers to the “D” isomeric form of a peptide. The amino acid residues described herein are preferred to be in the “L” isomeric form, however, residues in the “D” isomeric form can be substituted for any L-amino acid residue, as long as the desired functional is retained by the peptide.

The term “monomer” as used herein may also be referred to as “peptide monomer,” “peptide monomer molecule,” or “monomer peptide.” The term “monomer” indicates a single sequence of two or more amino acids joined together by peptide bonds.

Unless otherwise indicated, the term “NH₂,” as used herein, refers to the free amino group present at the amino terminus of a polypeptide. The term “OH,” as used herein, refers to the free carboxy group present at the carboxy terminus of a peptide. Further, the term “Ac,” as used herein, refers to Acetyl protection through acylation of the N-terminus of a polypeptide. Where indicated, “NH₂” refers to a free amino group side chain of an amino acid. Where indicated, the term “Ac,” as used herein refers to acylation of an amino acid with NH₂ group.

The term “N(alpha)Methylation”, as used herein, describes the methylation of the alpha amine of an amino acid, also generally termed as an N-methylation.

The term “peptide,” as used herein, refers broadly to a structure comprising a sequence of two or more amino acids joined together by peptide bonds. In particular embodiments, it refers to a sequence of two or more amino acids joined together by peptide bonds. It should be understood that this term does not connote a specific length of a polymer of amino acids, nor is it intended to imply or distinguish whether the polypeptide is produced using recombinant techniques, chemical or enzymatic synthesis, or is naturally occurring. The term “peptide”, as used generically herein, includes both peptide monomers and peptide dimers.

The term “pharmaceutically acceptable salt,” as used herein, represents salts or zwitterionic forms of the compounds of the invention, which are water or oil-soluble or dispersible, which are suitable for treatment of diseases without undue toxicity, irritation, and allergic response; which are commensurate with a reasonable benefit/risk ratio, and which are effective for their intended use. The salts can be prepared during the final isolation and purification of the compounds or separately by reacting an amino group with a suitable acid. Representative acid addition salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, fumarate, hydrochloride, hydrobromide, hydroiodide, 2-hydroxyethansulfonate (isethionate), lactate, maleate, mesitylenesulfonate, methanesulfonate, naphthylenesulfonate, nicotinate, 2-naphthalenesulfonate, oxalate, pamoate, pectinate, persulfate, 3-phenylproprionate, picrate, pivalate, propionate, succinate, tartrate, trichloroacetate, trifluoroacetate, phosphate, glutamate, bicarbonate, para-toluenesulfonate, and undecanoate. Also, amino groups in the compounds of the present invention can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric.

The term “placebo” means an inactive substance that has no therapeutic effect and look the same as, and is given the same way as, the compound of formula (I) being tested.

The term “receptor,” as used herein, refers to chemical groups of molecules on the cell surface or in the cell interior that have an affinity for a specific chemical group or molecule. Binding between compound of formula (I) or pharmaceutically acceptable salt thereof and targeted integrins can provide useful diagnostic tools.

As used herein, “remission” refers to the percentage of patients that are cured or obtain remission or complete resolution of a condition in response to a given treatment.

The term “thioether,” as used herein, refers to a cyclized, covalent bond formed between an upstream amino acid or aromatic acid group, and a downstream sulfur-containing amino acid, or isostere thereof, i.e., a C—S bond.

The term “ulcerative colitis” refers to A chronic, inflammatory bowel disease that causes inflammation in the digestive tract, specifically the colon and/or rectum. Ulcerative colitis is usually only in the innermost lining of the large intestine (colon) and rectum. Forms range from mild to severe. Having ulcerative colitis puts a patient at increased risk of developing colon cancer. Symptoms include rectal bleeding, bloody diarrhea, abdominal cramps, and pain. Mild-to-moderate ulcerative colitis is defined as patients with fewer than four to six bowel movements per day, mild or moderate rectal bleeding, absence of constitutional symptoms, low overall inflammatory burden, and absence of features suggestive of high inflammatory activity. Severe ulcerative colitis refers to severe bouts of bloody diarrhea, belly cramps, pain, fatigue, and major weight loss. It affects your entire colon and causes severe pain, heavy diarrhea, bleeding, and fever.

### Peptide Biological Activity

In certain embodiments, the compound of formula (I) or a pharmaceutically acceptable salt thereof has increased affinity for α4β7 binding, increased selectivity against α4=1, and increased stability in simulated intestinal fluid (SIF) as well as in gastric environment under reduced conditions. The compound of formula (I) or a pharmaceutically acceptable salt thereof demonstrates high binding affinity with α4β7, thereby preventing binding between α4β7 and the MAdCAM1 ligand. Accordingly, the compound of formula (I) or a pharmaceutically acceptable salt thereof have shown to be effective in eliminating and/or reducing the inflammation process in various experiments.

The compound of formula (I) or a pharmaceutically acceptable salt thereof binds or associates with the α4β7 integrin to disrupt or block binding between α4β7 and the MAdCAM1 ligand. In certain embodiments, the compound of formula (I) or a pharmaceutically acceptable salt thereof inhibits or reduces binding between α4β7 and the MAdCAM1 ligand. In certain embodiments, a compound of formula (I) or a pharmaceutically acceptable salt thereof reduces binding of α4β7 and the MAdCAM1 ligand by at least 20%, at least 30%, at least 40%, at least 50%, at least 60%, at least 70%, at least 80%, or at least 90% as compared to a negative control peptide. Methods of determining binding are known in the art and described herein, and include ELISA assays, for example.

In certain embodiments, a compound of formula (I) or a pharmaceutically acceptable salt thereof has an IC50 of <500 nM, <250 nM, <100 nM, <50 nM, <25 nM, or <10 nM. Methods of determining activity are known in the art and include any of those described in the accompanying Examples.

In some embodiments, a compound of formula (I) or a pharmaceutically acceptable salt thereof has a half-life of greater than 180 minutes when exposed to simulated intestinal fluids (SIF). The compound of formula (I) or a pharmaceutically acceptable salt thereof comprises a half-life from approximately 1 minute to approximately 180 minutes. Similarly, the compound of formula (I) or a pharmaceutically acceptable salt thereof is stable to gastric environment under reduced conditions with half-life>120 min when tested in DTT (Dithiothreitol) assay.

In certain embodiments, the compound of formula (I) or a pharmaceutically acceptable salt thereof has increased stability, increased gastrointestinal stability, and/or increased stability in stimulated intestinal fluid (SIF), as compared to a control peptide. In particular embodiments, a control peptide is a peptide having the identical or a highly related amino acid sequence (e.g., >90% sequence identity) as the compound of formula (I) or a pharmaceutically acceptable salt thereof, but which does not form a cyclized structure through a thioether bond. In some embodiments relating to the compound of formula (I) or a pharmaceutically acceptable salt thereof, the control peptide is not dimerized. In particular embodiments, the only difference between the compound of formula (I) or a pharmaceutically acceptable salt thereof and the control peptide is that the compound of formula (I) or a pharmaceutically acceptable salt thereof comprises one or more amino acid substitutions that introduce one or more amino acid residues into the peptide, wherein the introduced residue(s) forms a thioether bond with another residue in the peptide.

Methods of determining the stability of a peptide are known in the art. In certain embodiments, the stability of a peptide (e.g. a compound of formula (I) or a pharmaceutically acceptable salt thereof) is determined using an SIF assay, e.g., as described in the accompanying Examples. In particular embodiments, a peptide monomer or dimer molecule of the present invention has a half-life under a given set of conditions (e.g., temperature) of greater than 1 minute, greater than 10 minutes, greater than 20 minutes, greater than 30 minutes, greater than 60 minutes, greater than 90 minutes, greater than 120 minutes, greater than 3 hours, or greater than four hours when exposed to SIF. In certain embodiments, the temperature is about 25° C., about 4° C., or about 37° C., and the pH is a physiological pH, or a pH about 7.4.

In some embodiments, the half-life is measured in vitro using any suitable method known in the art, e.g., in some embodiments, the stability of the compound of formula (I) or a pharmaceutically acceptable salt thereof is determined by incubating the peptide with pre-warmed human serum (Sigma) at 37° C. Samples are taken at various time points, typically up to 24 hours, and the stability of the sample is analyzed by separating the compound of formula (I) or a pharmaceutically acceptable salt thereof from the serum proteins and then analyzing for the presence of the compound of formula (I) or a pharmaceutically acceptable salt thereof of interest using LC-MS.

In certain embodiments, the compound of formula (I) or a pharmaceutically acceptable salt thereof inhibit or reduce α4β7-mediated inflammation. In related embodiments, the compound of formula (I) or a pharmaceutically acceptable salt thereof inhibits or reduces α4β7-mediated secretion or release of one or more cytokines (including any disclosed herein) by T cells, e.g., T cells in the GI mucosa responding to MAdCAM1. Methods of determining inhibition of cytokine secretion and inhibition of signaling molecules are known in the art.

In certain embodiments, the compound of formula (I) or a pharmaceutically acceptable salt thereof demonstrates increased binding selectivity. In certain instances, the compound of formula (I) or a pharmaceutically acceptable salt thereof binds to α4β7 with at least a two-fold, three-fold, five-fold, or ten-fold greater affinity than the monomers or dimers bind to α4β1.

In some embodiments, the compound of formula (I) or a pharmaceutically acceptable salt thereof demonstrates increased potency as a result of substituting various natural amino acyl residues with N-methylated analog residues. In particular embodiments, potency is measured as IC50 of binding to α4β7, e.g., determined as described herein, while in some embodiments, potency indicates functional activity, e.g., according to a cell adhesion assay.

### Methods of Manufacture

The peptides (e.g., peptide monomers or peptide dimers) as disclosed herein may be synthesized by techniques that are known to those skilled in the art, e.g., as disclosed in PCT Application Publication Nos. WO 2014/059213, WO 2014/165448, WO 2014/165449, WO 2015/176035, WO 2016/054411, or WO 2016/054445. Such techniques include the use of commercially available robotic protein synthesizers (e.g., Symphony multiplex peptide synthesizer from Protein Technologies). In some embodiments, novel peptide monomers or dimer subunits are synthesized and purified using techniques described herein.

### Methods of Treatment

In some embodiments, the present invention provides methods for treating an individual or subject afflicted with a condition or indication characterized by α4β7 integrin binding, e.g., to MAdCAM1, wherein the methods comprise providing or administering to the individual or subject an integrin antagonist, e.g., a compound of formula (I) or pharmaceutically acceptable salt thereof, described herein. In particular embodiments, subjects or individuals are mammals, e.g., humans or non-human mammals, such as a dog, cat or horse. It is understood that the integrin antagonist may be present in a pharmaceutical composition, e.g., any of those disclosed herein. It is further understood that other agents that inhibit disrupt α4β7 integrin or MAdCAM1 signaling, or α4β7 integrin binding, e.g., to MAdCAM1, may be used as alternatives to the antagonists disclosed herein.

In another embodiment, a method is provided for treating an individual or subject afflicted with a condition or indication characterized by inappropriate trafficking of cells expressing α4β7 to tissues comprising cells expressing MAdCAM, comprising administering to the individual or subject a pharmaceutical composition comprising a compound of formula (I) or pharmaceutically acceptable salt thereof, in an amount sufficient to inhibit (partially or fully) the trafficking of cells expressing α4β7 to tissues comprising cells expressing MAdCAM.

In a further embodiment, the present invention includes a method for treating a condition in a subject or individual in need thereof, wherein the condition is treatable by reducing the activity (partially or fully) of α4β7 in the subject, comprising providing or administering an α4β7-antagonist peptide dimer compound described herein and an immunomodulator, optionally Tofacitinib, to the subject. In particular embodiments, the condition is an inflammatory condition of the gastrointestinal system, e.g., an IBD.

In a further related embodiment, the present invention includes a method for treating a subject, e.g., a mammal or human, afflicted with a condition that is associated with a biological function of α4β7 integrin, comprising providing or administering to the subject a compound of formula (I) or pharmaceutically acceptable salt thereof in an amount sufficient to inhibit (partially or fully) the biological function of α4β7 integrin to tissues expressing MAdCAM. In particular embodiments, the subject is provided with an effective amount of the peptide dimer compound sufficient to at least partially inhibit the biological function of α4β7 to tissues expressing MAdCAM. In certain embodiments, the condition is an inflammatory bowel disease, for example, ulcerative colitis.

In additional embodiments, the invention includes a method of treating or preventing a disease or condition in a subject in need thereof, comprising providing or administering to the subject, e.g., a mammal, an effective amount of a compound of formula (I) or pharmaceutically acceptable salt thereof, wherein the disease or condition is selected from the group consisting of Inflammatory Bowel Disease (IBD) (including but not limited to adult IBD, pediatric IBD and adolescent IBD), ulcerative colitis, Crohn's disease, Celiac disease (nontropical Sprue), enteropathy associated with seronegative arthropathies, microscopic colitis, collagenous colitis, eosinophilic esophagitis and gastroenteritis, radiotherapy, chemotherapy, pouchitis (e.g., pouchitis resulting after proctocolectomy and ileoanal anastomosis), proctitis, gastrointestinal cancer such as MALT lymphoma, pancreatitis, insulin-dependent diabetes mellitus, metabolic syndrome (diabesity) mastitis, systemic mastocytosis, cholecystitis, cholangitis, pericholangitis, chronic bronchitis, chronic sinusitis, asthma, primary sclerosing cholangitis, human immunodeficiency virus (HIV) infection in the GI tract, eosinophilic asthma, gastritis, colitis, microscopic colitis, chemotherapy-induced diarrhea, and graft versus host disease (GVDH) (including intestinal GVDH). In particular embodiments of any of the methods of treatment described herein, the subject has been diagnosed with or is considered to be at risk of developing one of these diseases or conditions.

In particular embodiments of any of the methods of treatment described herein, the compound of formula (I) or pharmaceutically acceptable salt thereof (or pharmaceutical composition comprising the compound of formula (I) or pharmaceutically acceptable salt thereof) is administered to the individual by a form of administration selected from the group consisting of oral, intravenous, peritoneal, intradermal, subcutaneous, intramuscular, intrathecal, inhalation, vaporization, nebulization, sublingual, buccal, parenteral, rectal, vaginal, and topical.

In certain embodiments, the compound of formula (I) or pharmaceutically acceptable salt thereof comprises an increased half-life as compared to other peptides. In particular embodiments, the increased half-life is at least one day in vitro or in vivo. In certain embodiments wherein the increased half-life is equal to or greater than a period consistent with no more frequent than twice daily dosing in vivo, the α4β7 integrin antagonist peptide dimer compound is provided in a pharmaceutical preparation that is administered orally. In certain embodiments wherein the increased half-life is from approximately 12 hours to greater than 24 in vivo, the compound of formula (I) or pharmaceutically acceptable salt thereof is provided in a pharmaceutical preparation that is administered parenterally. In certain embodiments when the increased half-life is from approximately 12 hours to greater than 24 hours in vivo, the α4β7 integrin antagonist peptide dimer compound is provided in a pharmaceutical preparation that is administered orally.

In some embodiments, the invention provides a method whereby a compound of formula (I) or pharmaceutically acceptable salt thereof is administered to a subject or patient as a first treatment.

In another embodiment, the method further comprises administering to the subject a compound of formula (I) or pharmaceutically acceptable salt thereof in combination with an immunomodulator. In another embodiment, the immunomodulator is administered to the subject before and/or simultaneously with and/or after the compound of formula (I) or pharmaceutically acceptable salt thereof is administered to the subject.

As used herein, an “immunomodulator” is an agent capable of altering the immune response of a subject. In particular embodiments, an immunomodulator enhances an immune response and may be referred to as “immunostimulatory.” In other embodiments, an “immunomodulator” dampens an immune response and may be referred to as “immunosuppressive.” In particular embodiments of any of the compositions or methods disclosed herein, the immunomodulator is immunostimulatory or immunosuppressive.

In one embodiment, the immunomodulator is at least one selected from the group consisting of azathioprine, cyclophosphamide, cyclosporine, hydroxychloroquine, leflunomide, methotrexate, mycophenolate, sulfasalazine, apremilast, tofacitinib, azathioprine, mercaptopurine, steroids, cortisone, cortisone acetate, dexamethasone, hydrocortisone, hydrocortisone acetate, methylprednisolone, prednisolone, prednisone, tixocortol pivalate, triamcinolone acetonide, triamcinolone alcohol, mometasone, amcinonide, budesonide, desonide, fluocinonide, fluocinolone acetonide, halcinonide, betamethasone, betamethasone sodium phosphate, dexamethasone, dexamethasone sodium phosphate, fluocortolone, hydrocortisone-17-valerate, acleometasone dipropionate, betamethasone valerate, betamethasone dippropionate, prednicarbate, clobetasone-17-butyrate, clobetasol-17-propionate, fluocortilone caproate, fluocortolone pivalate, and fluprednidene acetate, hydrocortisone-17-butyrate, 17-aceponate, 17-buteprate, and prednicarbate.

In one embodiment, the immunomodulator is a JAK1, JAK2, or JAK3 inhibitor.

In one embodiment, the immunomodulator is filgotinib, upadacitinib, tofacitinib, or baricitinib.

In other embodiments, the immunomodulator comprises an anti-inflammatory agent.

In another embodiment, the second treatment or active agent (which may be present in a pharmaceutical composition) comprises an agent selected from the group consisting of non-steroidal anti-inflammatory drugs and steroids. In another embodiment, the method comprises administering to the subject a third therapeutic agent.

Surprisingly, the compound of formula (I) can be used to target a plasma concentration to achieve a better therapeutic effect. For example, therapeutic drug monitoring can be used to achieve a desired therapeutic effect or clinical efficacy.

In one aspect, the present disclosure provides methods of treating IBDs by inhibiting α4β7 integrin using a peptide compound of formula (I):

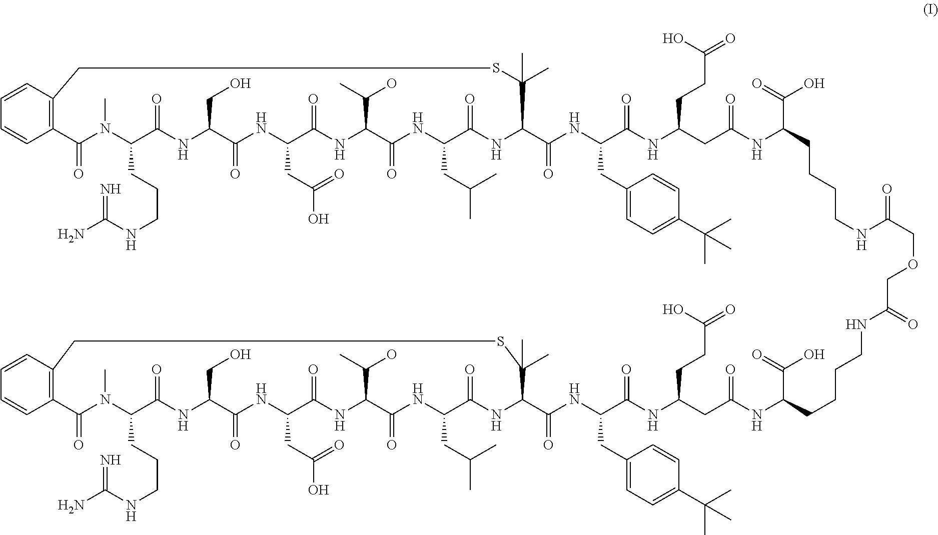 [SMILES: CC(C)C[C@@H]1NC(=O)[C@H](C(C)O)NC(=O)[C@H](CC(=O)O)NC(=O)[C@H](CO)NC(=O)[C@H](CCCNC(=N)N)N(C)C(=O)c2ccccc2CSC(C)(C)[C@@H](C(=O)N[C@@H](Cc2ccc(C(C)(C)C)cc2)C(=O)N[C@@H](CCC(=O)O)CC(=O)N[C@H](CCCCNC(=O)COCC(=O)NCCCC[C@@H](NC(=O)C[C@H](CCC(=O)O)NC(=O)[C@H](Cc2ccc(C(C)(C)C)cc2)NC(=O)[C@H]2NC(=O)[C@H](CC(C)C)NC(=O)[C@H](C(C)O)NC(=O)[C@H](CC(=O)O)NC(=O)[C@H](CO)NC(=O)[C@H](CCCNC(=N)N)N(C)C(=O)c3ccccc3CSC2(C)C)C(=O)O)C(=O)O)NC1=O]

or a pharmaceutically salt thereof or a pharmaceutical composition comprising a compound of formula (I) or a pharmaceutically acceptable salt thereof. The method comprises orally administering to the subject a compound of formula (I) or a pharmaceutically acceptable salt thereof or a pharmaceutical composition comprising a compound of formula (I) or a pharmaceutically acceptable salt thereof, wherein the subject has a plasma concentration of the compound at from about 0.1 ng/mL to about 4 ng/mL and wherein the administering compound of formula (I) to the subject achieves a clinical remission rate of at least about 1%, 2%, 3%, 4%, 5% or 6% higher relative to a clinical remission rate from placebo. Examples of IBDs treatable include, but are not limited to, ulcerative colitis and Crohn's disease. In particular embodiments, the subject has the plasma concentration of the compound of from about 0.1 ng/mL to about 4 ng/mL at some time or during some time period following administration of the compound or pharmaceutically acceptable salt thereof. In particular embodiments, this range of plasma concentration is achieved within about 1 hour, about 2 hours, about 3 hours, about 4 hours, about 5 hours, about 6 hours, about 7 hours about 8 hours, about 10 hours, or about 12 hours following administration of the compound or pharmaceutically acceptable salt thereof. In certain embodiments, this range of plasma concentration is maintained throughout a course of treatment with the compound or pharmaceutically acceptable salt thereof. In particular embodiments, this range is maintained for a majority of a course of treatment with the compound of formula (I) or pharmaceutically acceptable salt thereof. For example, in certain embodiments, over a course of treatment, the plasma concentration may dip below or rise above this range, and the dosage may be adjusted higher or lower to once again achieve the desired plasma concentration range. In particular embodiments of the methods, the plasma concentration of the compound of Formula (I) or pharmaceutically acceptable salt thereof is from about 0.1 ng/mL to about 4 ng/mL, about 0.1 ng/mL to about 2 ng/mL, about 0.1 ng/mL to about 1 ng/mL, or about 0.2 ng/mL to about 1.0 ng/mL, at some time or during some time period following administration of the compound or pharmaceutically acceptable salt there, or over the course of treatment (or a majority of the course of treatment).

In one aspect, the disclosure provides a method of treating an IBD, e.g., ulcerative colitis or Crohn's disease, comprising administering to a subject in need thereof an amount of the compound of Formula (I) or a pharmaceutically acceptable salt thereof, at a dose and/or dosing regimen effective to achieve and/or maintain the subject's plasma concentration of the compound Formula (I) or pharmaceutically acceptable salt thereof from about 0.1 ng/mL to about 4 ng/mL over the course of treatment (or the majority of the course of treatment). In certain embodiments, the subject is administered an amount of the compound of Formula (I) or pharmaceutically acceptable salt thereof effective to achieve and/or maintain the subject's plasma concentration of the compound Formula (I) or pharmaceutically acceptable salt thereof in a range of from about 0.1 ng/mL to about 4 ng/mL, about 0.1 ng/mL to about 2 ng/mL, about 0.1 ng/mL to about 1 ng/mL, or about 0.2 ng/mL to about 1.0 ng/mL, at some time or during some time period following administration of the compound or pharmaceutically acceptable salt there, or over the course of treatment (or a majority of the course of treatment).

In another aspect, the disclosure provides a method of treating an IBD, e.g., ulcerative colitis or Crohn's disease, comprising:
(a) administering to a subject in need thereof a first amount of the compound of Formula (I) or a pharmaceutically acceptable salt thereof;
(b) determining a first plasma concentration of the compound of Formula (I) or pharmaceutically acceptable salt thereof in the subject;
(c) administering to the subject a second amount of the compound of Formula (I) or pharmaceutically acceptable salt thereof, wherein:
   (i) if the first plasma concentration is in a range of from about 0.1 ng/mL to about 4 ng/mL, about 0.1 ng/mL to about 2 ng/mL, about 0.1 ng/mL to about 1 ng/mL, or about 0.2 ng/mL to about 1.0 ng/mL, then the second amount administered (step (c)) is about the same as the first amount administered (step (a);
   (ii) if the first plasma concentration is lower than a range of from about 0.1 ng/mL to about 4 ng/mL, about 0.1 ng/mL to about 2 ng/mL, about 0.1 ng/mL to about 1 ng/mL, or about ng/mL to about 1.0 ng/mL, then the second amount administered (step (c)) is greater than the first amount administered (step (a); and
   (iii) if the first plasma concentration is greater than a range of from about 0.1 ng/mL to about 4 ng/mL, about 0.1 ng/mL to about 2 ng/mL, about 0.1 ng/mL to about 1 ng/mL, or about ng/mL to about 1.0 ng/mL, then the second amount administered (step (c)) is less than the first amount administered (step (a). Steps (a)-(c) or steps (b)-(c) may be repeated one time or more than once over the course of a treatment regimen, e.g., with second, third, etc. plasma concentrations determined, and third, fourth, etc. amounts of the compound of Formula (I) or pharmaceutically acceptable salt thereof administered. In certain embodiments, the method maintains the subject's plasma concentration of the compound Formula (I) or pharmaceutically acceptable salt thereof in a range of from about 0.1 ng/mL to about 4 ng/mL, about 0.1 ng/mL to about 2 ng/mL, about 0.1 ng/mL to about 1 ng/mL, or about 0.2 ng/mL to about 1.0 ng/mL, at some time or during some time period following administration of the compound or pharmaceutically acceptable salt there, or over the course of treatment (or a majority of the course of treatment).

In some embodiments of methods of treating IBDs as disclosed herein, the compound of formula (I) is administered to the subject at a dose from about 50 mg to about 1000 mg per day as divided doses or as a once daily dose.

In some embodiments of methods of treating IBDs as disclosed herein, the compound of formula (I) is administered to the subject at a dose of about 50, 62.5, 75, 87.5, 100.0, 112.5, 125.0, 137.5, 150.0, 162.5, 175, 187.5, 200.0, 212.5, 225.0, 237.5, 250.0, 262.5, 275, 287.5, 300.0, 312.5, 325.0, 337.5, 350.0, 362.5, 375, 387.5, 400.0, 412.5, 425.0, 437.5, 450.0, 462.5, 475, 487.5, 500.0, 512.5, 525, 537.5, 550, 562.5, 575, 587.5, 600, 612.5, 625, 637.5, 650, 662.5, 675, 687.5, 700, 712.5, 725, 737.5, 750, 762.5, 775, 787.5,800, 812.5, 825, 837.5, 850, 862.5, 875, 887.5, 900, 912.5, 925, 937.5, 950, 962.5, 975, 987.5 or 1000 mg.

In some embodiments of the methods disclosed herein, the peptide dimer compound or pharmaceutically acceptable salt thereof is administered to the subject at a dose of about 100.0, 112.5, 125.0, 137.5, 150.0, 162.5, 175, 187.5, 200.0, 212.5, 225.0, 237.5, 250.0, 262.5, 275, 287.5, 300.0, 312.5, 325.0, 337.5, 350.0, 362.5, 375, 387.5, 400.0, 412.5, 425.0, 437.5, 450.0, 462.5, 475, 487.5, or 500.0 mg. In one embodiment, the peptide dimer compound or pharmaceutically acceptable salt thereof is administered to the subject at a dose of about 150 mg or about 450 mg. In certain embodiments, the dose is administered to the subject once or twice daily.

In some embodiments of the methods as disclosed herein, the administering compound of formula (I) to the subject achieves a clinical remission rate of at least about 6%, 7%, 8%, 9%, 10%, 11%, 12%, 13%, 14%, 15%, 16%, 20%, 21%, 22%, 23%, 24%, 25%, 27.5%, 30%, 40%, 50%, 80%, 90%, 100%, 200%, 300%, 400%, 500%, 600%, 700%, 800%, 900%, 1000% higher relative to a clinical remission rate from placebo.

In some embodiments of methods of treating IBDs as disclosed herein, administering compound of formula (I) to the subject achieves a clinical remission rate from about 13% to about 40% higher relative to the clinical remission rate from placebo.

In some embodiments of methods of treating IBDs as disclosed herein, administering compound of formula (I) to the subject achieves a clinical remission rate at least about 40% higher relative to the histologic remission rate from placebo.

In some embodiments of methods of treating IBDs as disclosed herein, administering compound of formula (I) to the subject achieves a histologic remission rate from about 20% to about 37% higher relative to the clinical remission rate from placebo.

In some embodiments of methods of treating IBDs as disclosed herein, administering compound of formula (I) to the subject achieves a histologic remission rate of at least 20% higher relative to the histologic remission rate from placebo.

In some embodiments of the methods as disclosed herein, the administering compound of formula (I) to the subject achieves a histologic remission rate of at least about 20%, 21%, 22%, 23%, 24%, 25%, 27.5%, 30%, 40%, 50%, 60%, 70%, 80%, 90%, 100%, 200%, 300%, 400%, 500%, 600%, 700%, 800%, 900%, 1000% higher relative to a histologic remission rate from placebo.

In another aspect, the present disclosure provides a method of optimizing or improving therapeutic efficacy for treating IBD in a subject in need thereof, the method comprising orally administering a compound of formula (I):

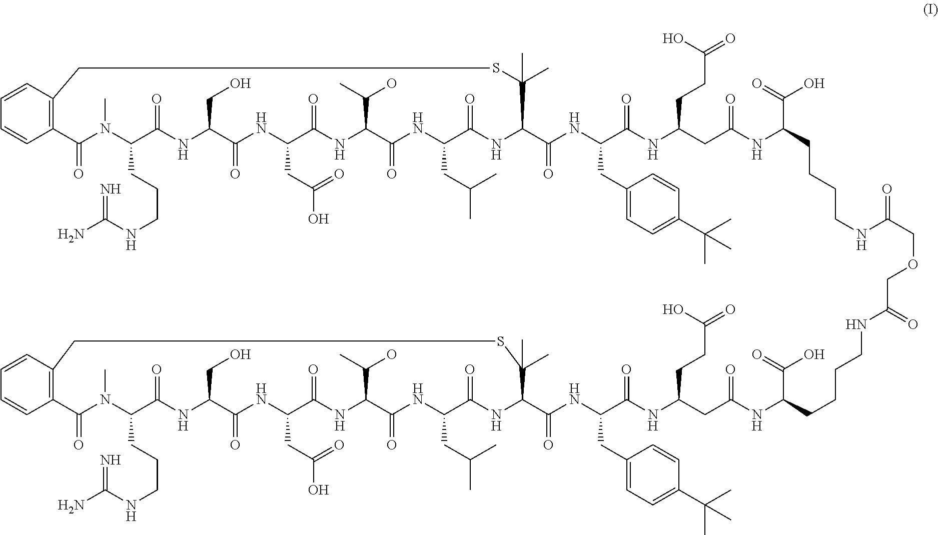 [SMILES: CC(C)C[C@@H]1NC(=O)[C@H](C(C)O)NC(=O)[C@H](CC(=O)O)NC(=O)[C@H](CO)NC(=O)[C@H](CCCNC(=N)N)N(C)C(=O)c2ccccc2CSC(C)(C)[C@@H](C(=O)N[C@@H](Cc2ccc(C(C)(C)C)cc2)C(=O)N[C@@H](CCC(=O)O)CC(=O)N[C@H](CCCCNC(=O)COCC(=O)NCCCC[C@@H](NC(=O)C[C@H](CCC(=O)O)NC(=O)[C@H](Cc2ccc(C(C)(C)C)cc2)NC(=O)[C@H]2NC(=O)[C@H](CC(C)C)NC(=O)[C@H](C(C)O)NC(=O)[C@H](CC(=O)O)NC(=O)[C@H](CO)NC(=O)[C@H](CCCNC(=N)N)N(C)C(=O)c3ccccc3CSC2(C)C)C(=O)O)C(=O)O)NC1=O]

or a pharmaceutically acceptable salt thereof or a pharmaceutical composition comprising a compound of formula (I) or a pharmaceutically acceptable salt thereof to the subject, wherein the plasma concentration of the compound of formula (I) in the subject is maintained at a predetermined concentration to achieve a desired therapeutic effect.

In some embodiments, the compound of formula (I) is used to target a plasma concentration to achieve a better or improved therapeutic effect. For example, therapeutic drug monitoring is used to achieve a desired therapeutic effect

In some embodiments of the methods as disclosed herein, the plasma concentration or target plasma concentration or predetermined plasma concentration of the compound of formula (I) is between about 0.2 and about 0.7 ng/mL; about 0.1 and about 0.5; about 0.1 and about 0.85; about 0.3 and about 0.7; about 0.4 and about 0.85; about 0.7 and 0.9; or about 0.8 and about 2.0 ng/mL. In one embodiment, the plasma concentration or target plasma concentration or predetermined plasma concentration of the compound of formula (I) is at least about 2 ng/mL.

In a preferred embodiment, the plasma concentration or target plasma concentration or predetermined plasma concentration of the compound of formula (I) is between about 0.3 and about 0.7 ng/mL. In one embodiment, the plasma concentration or target plasma concentration or predetermined plasma concentration of the compound of formula (I) is between about 0.3 and 0.8 ng/mL. In another embodiment, the plasma concentration or target plasma concentration or predetermined plasma concentration of the compound of formula (I) is between about 0.2 and about 0.3 ng/mL.

In certain embodiments, the plasma concentration or target plasma concentration or predetermined plasma concentration of the compound of formula (I) is about 0.1, 0.15, 0.2, 0.3, 0.35, 0.4, 0.45, 0.5, 0.55, 0.6, 0.65, 0.7, 0.75, 0.8, 0.85, 0.9, 0.95, 1.0, 1.1, 1.2, 1.3, 1.4, 1.5, 1.6, 1.7, 1.8, 1.9 or 2.0 ng/mL. In one embodiment, the plasma concentration or target plasma concentration or predetermined plasma concentration of the compound of formula (I) is about 0.2 or about 0.7 ng/mL. In another embodiment, the plasma concentration or target plasma concentration or predetermined plasma concentration of the compound of formula (I) is about 2.1 or about 3.3 ng/mL.

In some embodiments of the methods as disclosed herein, the plasma concentration is maintained by determining the plasma concentration of the compound of formula (I) in the subject; and adjusting administering the compound of formula (I) or a pharmaceutically acceptable salt thereof to achieve a desired therapeutic effect. The target plasma concentration of the compound of formula (I) can be achieved by changing the dosage regimen of the compound administered to the subject. For example, changing the dose, schedule, and frequency of the administration of the compound of formula (I).

In some embodiments of the methods as disclosed herein, the compound of formula (I) or a pharmaceutically acceptable salt thereof is administered twice daily.

In some embodiments of the methods as disclosed herein, the subject is a human patient.

In some embodiments of the methods as disclosed herein, the IBD treatable is ulcerative colitis. In another embodiment, the IBD treatable is moderate to severe ulcerative colitis. In another embodiment, the IBD is Crohn's disease.

In particular embodiments, the disclosure provides a unit dosage form of a compound of formula (I), which comprises about any of 5, 6, 7, 8, 9, 10, 12.5, 25.0, 37.5, 50.0, 62.5, 75, 87.5, 100.0, 112.5, 125.0, 137.5, 150.0, 162.5, 175, 187.5, 200.0, 212.5, 225.0, 237.5, 250.0, 262.5, 275, 287.5, 300.0, 312.5, 325.0, 337.5, 350.0, 362.5, 375, 387.5, 400.0, 412.5, 425.0, 437.5, 450.0, 462.5, 475, 487.5, or 500.0 mg of the compound. In some embodiments, the unit dosage form comprises about any of 12.5, 25.0, 37.5, 50.0, 62.5, 75, 87.5, 100.0, 112.5, 125.0, 137.5, 150.0, 162.5, 175, 187.5, 200.0, 212.5, 225.0, 237.5, 250.0, 262.5, 275, 287.5, 300.0, 350.0, 400.0, 450.0, or 500.0 mg. In some embodiments, the unit dosage form comprises about any of 70, 75, 80, 85, 90, 95, 100, 105, 110, 115, 120, 125, or 130 mg. In some embodiments, the unit dosage form comprises about any of 85, 90, 95, 100, 105, 110, or 115 mg. In some embodiments, the unit dosage form comprises about any of 95, 100, or 105 mg. In some embodiments, the unit dosage form comprises about 100 mg. In some embodiments, the unit dosage form comprises from about 100 to 500 mg. In some embodiments, the unit dosage form comprises about any of 100, 150, 200, 250, 300, 250, 400, 450, or 500 mg. In some embodiments, unit dosage form comprises about 450 mg or about 150 mg. In particular embodiments, the unit dosage form comprises a pharmaceutical composition comprising the peptide dimer compound, e.g., any of those disclosed herein. In particular embodiments, it is formulated for oral administration, e.g., as a tablet. In certain embodiments, it is formulated for rectal administration, e.g., as a suppository. In some embodiments, the unit dosage form comprises about 450 mg or about 150 mg of a compound of formula (I) or a pharmaceutically acceptable salt thereof. In particular embodiments, the unit dosage form comprises a pharmaceutical composition comprising a compound of formula (I) or a pharmaceutically acceptable salt thereof.

### Pharmaceutical Compositions

In particular embodiments, the peptide compound of formula (I) is present in a pharmaceutical composition further comprising one or more pharmaceutically acceptable diluents, carriers, or excipients. In particular embodiments, they are formulated as a liquid or solid. In particular embodiments, they are formulated as a tablet or capsule, or as a liquid suspension. Some embodiments of the present invention further provide a method for treating an individual with an α4β7 integrin antagonist compound of formula (I) or pharmaceutically acceptable salt thereof of the present invention that is suspended in a sustained-release matrix. A sustained-release matrix, as used herein, is a matrix made of materials, usually polymers, which are degradable by enzymatic or acid-base hydrolysis or by dissolution. Once inserted into the body, the matrix is acted upon by enzymes and body fluids. A sustained-release matrix desirably is chosen from biocompatible materials such as liposomes, polylactides (polylactic acid), polyglycolide (polymer of glycolic acid), polylactide co-glycolide (copolymers of lactic acid and glycolic acid) polyanhydrides, poly(ortho)esters, polypeptides, hyaluronic acid, collagen, chondroitin sulfate, carboxylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. On particular biodegradable matrix is a matrix of one of either polylactide, polyglycolide, or polylactide co-glycolide (co-polymers of lactic acid and glycolic acid).

In some embodiments, the disclosure provides a pharmaceutical composition for oral delivery. The various embodiments and compound of formula (I) or pharmaceutically acceptable salt thereof compositions of may be prepared for oral administration according to any of the methods, techniques, and/or delivery vehicles described herein. Further, one having skill in the art will appreciate that the compound of formula (I) or pharmaceutically acceptable salt thereof compositions of the instant invention may be modified or integrated into a system or delivery vehicle that is not disclosed herein yet is well known in the art and compatible for use in oral delivery of small peptide molecules.

Oral dosage forms or unit doses compatible for use with the compound of formula (I) may include a mixture of peptide active drug components, and nondrug components or excipients, as well as other non-reusable materials that may be considered either as an ingredient or packaging. Oral compositions may include at least one of a liquid, a solid, and a semi-solid dosage forms. In some embodiments, an oral dosage form is provided comprising an effective amount of a compound of formula (I) or pharmaceutically acceptable salt thereof described herein, wherein the dosage form comprises at least one of a pill, a tablet, a capsule, a gel, a paste, a drink, and a syrup. In some instances, an oral dosage form is provided that is designed and configured to achieve delayed release of the thioether compound of formula (I) or pharmaceutically acceptable salt thereof in the small intestine of the subject.

In one embodiment, an oral pharmaceutical composition comprising the compound of formula (I) comprises an enteric coating that is designed to delay release of the compound of formula (I) or pharmaceutically acceptable salt thereof in the small intestine. In some instances it is preferred that a pharmaceutical composition of the instant invention comprise an enteric coat that is soluble in gastric juice at a pH of about 5.0 or higher. In at least one embodiment, a pharmaceutical composition is provided comprising an enteric coating comprising a polymer having dissociable carboxylic groups, such as derivatives of cellulose, including hydroxypropylmethyl cellulose phthalate, cellulose acetate phthalate and cellulose acetate trimellitate and similar derivatives of cellulose and other carbohydrate polymers.

In one embodiment, a pharmaceutical composition comprising a compound of formula (I) is provided in an enteric coating, the enteric coating being designed to protect and release the pharmaceutical composition in a controlled manner within the lower gastrointestinal system of a subject, and to avoid systemic side effects. In addition to enteric coatings, the compound of formula (I) may be encapsulated, coated, engaged or otherwise associated within any compatible oral drug delivery system or component. For example, in some embodiments a compound of formula (I) or pharmaceutically acceptable salt thereof of the present invention is provided in a lipid carrier system comprising at least one of polymeric hydrogels, nanoparticles, microspheres, micelles, and other lipid systems.

To overcome peptide degradation in the small intestine, some implementations of the present invention comprise a hydrogel polymer carrier system in which a compound of formula (I) or pharmaceutically acceptable salt thereof in accordance with the present invention is contained, whereby the hydrogel polymer protect the peptide from proteolysis in the small intestine. The compound of formula (I) or pharmaceutically acceptable salt thereof s may further be formulated for compatible use with a carrier system that is designed to increase the dissolution kinetics and enhance intestinal absorption of the peptides. These methods include the use of liposomes, micelles and nanoparticles to increase GI tract permeation of peptides.

Various bioresponsive systems may also be combined with one or more compound of formula (I) or pharmaceutically acceptable salt thereof of the present invention to provide a pharmaceutical agent for oral delivery. In some embodiments, a compound of formula (I) or pharmaceutically acceptable salt thereof of the instant invention is used in combination with a bioresponsive system, such as hydrogels and mucoadhesive polymers with hydrogen bonding groups (e.g., PEG, poly(methacrylic) acid [PMAA], cellulose, Eudragit®, chitosan and alginate) to provide a therapeutic agent for oral administration. Other embodiments include a method for optimizing or prolonging drug residence time for a compound of formula (I) or pharmaceutically acceptable salt thereof disclosed herein, wherein the surface of the compound of formula (I) or pharmaceutically acceptable salt thereof is modified to comprise mucoadhesive properties through hydrogen bonds, polymers with linked mucins or/and hydrophobic interactions. These modified compound of formula (I) or pharmaceutically acceptable salt thereof s may demonstrate increase drug residence time within the subject, in accordance with a desired feature of the invention. Moreover, targeted mucoadhesive systems may specifically bind to receptors at the enterocytes and M-cell surfaces, thereby further increasing the uptake of particles containing the compound of formula (I) or pharmaceutically acceptable salt thereof.

Other embodiments comprise a method for oral delivery of a peptide molecule described herein wherein the peptide molecule is used in combination with permeation enhancers that promote the transport of the peptides across the intestinal mucosa by increasing paracellular or transcellular permeation. For example, in one embodiment a permeation enhancer is combined with a peptide molecule described herein, wherein the permeation enhancer comprises at least one of a long-chain fatty acid, a bile salt, an amphiphilic surfactant, and a chelating agent. In one embodiment, a permeation enhancer comprising sodium N-[(hydroxybenzoyl)amino] caprylate is used to form a weak noncovalent association with the peptide molecule of the instant invention, wherein the permeation enhancer favors membrane transport and further dissociation once reaching the blood circulation. In another embodiment, a peptide molecule is conjugated to oligoarginine, thereby increasing cellular penetration of the peptide into various cell types. Further, in at least one embodiment a noncovalent bond is provided between a peptide molecule described herein and a permeation enhancer selected from the group consisting of a cyclodextrin (CD) and a dendrimers, wherein the permeation enhancer reduces peptide aggregation and increasing stability and solubility for the peptide molecule.

When used in at least one of the treatments or delivery systems described herein, a therapeutically effective amount of the compound of formula (I) may be employed in pure form or, where such forms exist, in pharmaceutically acceptable salt form. As used herein, a “therapeutically effective amount” of the compound of the invention is meant to describe a sufficient amount of the peptide molecule to treat an integrin-related disease, (for example, to reduce inflammation associated with IBD) at a desired benefit/risk ratio applicable to any medical treatment. It will be understood, however, that the total daily usage of the compounds and compositions of the present invention will be decided by the attending physician within the scope of sound medical judgment. The specific therapeutically effective dose level for any particular patient will depend upon a variety of factors including: a) the disorder being treated and the severity of the disorder; b) activity of the specific compound employed; c) the specific composition employed, the age, body weight, general health, sex and diet of the patient; d) the time of administration, route of administration, and rate of excretion of the specific compound employed; e) the duration of the treatment; f) drugs used in combination or coincidental with the specific compound employed, and like factors well known in the medical arts.

Alternatively, the compound of formula (I) may be administered as pharmaceutical compositions containing the compound of formula (I) in combination with one or more pharmaceutically acceptable excipients. A pharmaceutically acceptable carrier or excipient refers to a non-toxic solid, semi-solid or liquid filler, diluent, encapsulating material or formulation auxiliary of any type. The compositions may be administered parenterally, intracisternally, intravaginally, intraperitoneally, intrarectally, topically (as by powders, ointments, drops, suppository, or transdermal patch), rectally, or buccally. The term “parenteral” as used herein refers to modes of administration which include intravenous, intramuscular, intraperitoneal, intrasternal, subcutaneous, intradermal and intraarticular injection and infusion.

Compositions for rectal or vaginal administration are preferably suppositories which may be prepared by mixing the compounds of this invention with suitable non-irritating excipients or carriers such as cocoa butter, polyethylene glycol or a suppository wax which are solid at room temperature but liquid at body temperature and therefore melt in the rectum or vaginal cavity and release the active compound.

Total daily dose of the compositions of the invention to be administered to a human or other mammal host in single or divided doses may be in amounts, for example, from 0.0001 to 300 mg/kg body weight daily and more usually 1 to 300 mg/kg body weight.

### EXAMPLES

### Example 1

### Randomized, Double-Blind, Placebo-Controlled Study to Evaluate Safety and Efficacy of Oral Compound (I) in Subjects with Moderate to Severe Active Ulcerative Colitis

A phase 2 randomized, double-blind, placebo-controlled clinical study in human patients with moderate to severe ulcerative colitis is conducted to demonstrate safety, tolerability, and efficacy of treatment with oral Compound (I). The study also evaluates the pharmacokinetic (PK) and pharmacodynamics (PD) and biomarker responses to treatment with oral Compound (I).

### Study Design

This is a two-part study: Part 1 is a randomized, double-blind, placebo-controlled, parallel design 12-week induction treatment period in patients with moderate to severe active UC; and Part 2 is an extended treatment period of 40 weeks that will include subjects who successfully complete Part 1. Subjects who complete the Week 12 visit for Part 1 will be eligible to enter Part 2.

### Part 1: Induction Treatment Period (ITP)

Part 1 is a 12-week randomized, double-blind, placebo-controlled, parallel design study in adult subjects with moderate to severe active UC. Eligible subjects are randomized 1:1:1 to Compound (I) 450 mg twice daily (BID), Compound (I) 150 mg BID, or placebo BID. Subjects must have a biopsy-confirmed diagnosis of UC. To satisfy inclusion criteria, eligible subjects must have had a prior inadequate initial response, loss of response or intolerance to an older conventional therapy for UC (i.e., a corticosteroid, aminosalicylate or immunomodulator) or prior inadequate initial response, loss of response or intolerance to a newer biologic therapy (i.e., a TNFα antagonist or an IL12/23 antagonist). Subjects with a history of prior vedolizumab treatment will be excluded. Randomization will be stratified by prior failure to a TNFα antagonist or an IL-12/23 antagonist.

Eligible subjects satisfy the following inclusion criteria:
Male and female subjects age 18 (or the minimum country specific age of consent if >18) to 75 years;
Subject understands the study procedures and agrees to participate in the study by giving written informed consent;
Diagnosis of UC supported by appropriate documentation of biopsy results consistent with UC
Moderate to severe active UC; and
Demonstrated inadequate response, loss of response, or intolerance of at least 1 of oral aminosalicylates (5-ASAs), corticosteroids, immunomodulators, or a biologic (excluding vedolizumab),
   and eligible subjects do not meet the following exclusion criteria:
Subject with a current diagnosis of Crohn's disease (CD), indeterminate colitis (IC), microscopic colitis, ischemic colitis, radiation colitis;
History of colonic dysplasia other than completely removed low-grade dysplastic lesion;
History of active bacterial, viral, fungal or mycobacterial infection requiring hospitalization or IV antibiotic/anti-infective treatment within 4 weeks of screening or oral antibiotics/anti-infectives within 2 weeks of screening;
Prior treatment with vedolizumab, natalizumab, or any agent targeting the α4β7 or β1 integrin or planned during the study;
Positive stool test for C. difficile;
Chronic recurrent or serious infection;
Known primary or secondary immunodeficiency;
Pregnant or lactating female or considering becoming pregnant during the study or within 30 days after the last dose of study medication; and
History of any major neurological disorders.

Eligible subjects are randomized 1:1:1 to Compound (I) 450 mg twice daily (BID), Compound (I) 450 150 mg BID, or placebo BID.

### Part 2: Extended Treatment Period (ETP):

Subjects who complete the Week 12 visit for Part 1, including components of Adapted Mayo Score, will be eligible to enter Part 2. All Part 1 completers will be eligible to enter into Part 2, the extended treatment period, at the discretion of the investigator. Subjects will be assigned to the appropriate extended treatment arm in a blinded fashion. All subjects continuing into Part 2 will receive Compound (I).

### Numer of Subjects

Approximately 150 subjects will be randomized in this study.

### Treatment Assignment

### Part 1 (ITP)

Treatment assignment will be implemented using an IxRS. Eligible subjects will be stratified by prior biologic failure (yes/no) at randomization (Week 0) and randomized 1:1:1 to receive Compound (I) 450 mg BID, Compound (I) 150 mg BID, or placebo BID. Prior failure to a biologic is defined as failure to either a TNFα antagonist or an IL-12/23 antagonist.

### Part 2 (ETP)

All subjects who complete Week 12 visit and have a completed Adapted Mayo Score will receive Compound (I). Since enrollment and treatment will be ongoing in Part 1, treatment assignment in Part 2 will be implemented in a blinded fashion using an IxRS.
Subjects who receive Compound (I) 450 mg BID in Part 1 (induction): will be eligible to receive Compound (I) 450 mg BID.
Subjects who receive Compound (I) 150 mg BID in Part 1 (induction): Dose level assignment will depend on clinical response status. Clinical response is defined as a reduction≥30%, with a reduction in the RBS≥1 or an absolute subscore≤1. Adapted Mayo score is the sum of three components: RBS, SFS, and ESS from Mayo Score at Week 12
   Clinical responder: will be eligible to receive Compound (I) 150 mg BID.
   Non-responder: will be eligible will to receive Compound (I) 450 mg BID.

Subjects who receive placebo BID in Part 1 (induction): will be eligible to receive Compound (I) 150 mg BID.

### Test Product(s), Dose and Mode of Administration

Compound (I) (300 mg and 150 mg) and matching placebo tablets will be administered orally. Both Compound (I) strengths and placebo will have the same appearance.

### Study Treatments (Part 1)

Compound (I) 450 mg BID (1×300 mg tablet+1×150 mg tablet)
Compound (I) 150 mg BID (1×150 mg tablet+1 placebo tablet)
Placebo BID (2 placebo tablets)

### Study Treatments (Part 2)

Compound (I) 450 mg BID (1×300 mg tablet+1×150 mg tablet)
Compound (I) 150 mg BID (1×150 mg tablet+1 placebo tablet
   Compound (I) tablets and matching placebo tablets are administered orally. Subjects will be instructed to take the medication twice daily (approximately 8 to 12 hours apart) during the treatment period. The tablets must be taken with approximately 7 ounces/200 mL of water. Tablets should be swallowed whole without chewing. The subject should avoid consuming food within one hour before and after taking study medication.

### Outcome Analysis

Primary outcome measures include the proportion of subjects achieving clinical remission at Week 12 compared to placebo. Clinical remission is determined using the Adapted Mayo score (sum of 3 subscores from the Mayo score):
Stool frequency subscore (SFS)
Rectal bleeding subscore (RBS)
Endoscopic subscore (ESS)

Secondary outcome measures include a comparison between Compound (I) high-dose and low-dose individually to placebo:
Proportion of subjects with endoscopic improvement.
Proportion of subjects achieving endoscopic remission.
Proportion of subjects with histological improvement.
Proportion of subjects achieving histological remission.
Proportion of subjects with mucosal healing.

Other outcome measures include the proportion of subjects achieving clinical remission at Week 52. Clinical remission is determined using the Adapted Mayo score (sum of 3 subscores from the Mayo score):
Stool frequency subscore (SFS)
Rectal bleeding subscore (RBS)
Endoscopic subscore (ESS).

### Assessment of Efficacy

Efficacy is assessed, at least in part, based on the Mayo score. The Mayo score includes 4 components: Stool Frequency Subscore (SFS), Rectal Bleeding Subscore (RBS), Endoscopic Subscore (ESS) and Physician's Global Assessment (PGA). Each of the individual scores range from 0 to 3 with higher number indicating higher severity):
Complete Mayo Score is a sum of all 4 subscores (SFS, RBS, ESS and PGA) and ranges from 0 to 12 points.
Adapted Mayo Score is a sum of 3 subscores (SFS, RBS and ESS). The Adapted Mayo score ranges from 0 to 9 points.
Partial Mayo Score is a sum of 3 subscores (SFS, RBS and PGA) ranging from 0 to 9 points. Endoscopic subscore (ESS)≤1 (modified so that a score of 1 does not include friability).

### Results

It is expected that the treatment with any of the dosages of Compound (I) will be safe, and that treatment with either 450 mg BID or 150 mg BID will show statistically significant improvement in Complete Mayo Score, Adapted Mayo Score, and/or Partial Mayo Score as compared to treatment with placebo, thus demonstrating the effectiveness of these dosages of Compound (I) for treating ulcerative colitis.

### Pharmacokinetic, Pharmacodynamic Assessments

Blood Sample Collection Schedule in Each Study Period

Blood samples for measurement of Compound (I) plasma concentrations will be collected at 0 (within 1 hour pre-dose) and at 1, 2, and 4 hours post-dose on Week 0 (Study Day 1) and Week 12. A single pre-dose sample should be collected within 1 hour prior to the first dose of the day on each clinic visit.

Blood samples collected for Compound (I) plasma concentrations will be quantified using a validated assay. PK samples will be processed to obtain plasma and can be kept frozen before shipping to a central lab.

At selected sites only, blood samples will be collected from approximately 75 subjects and shipped to a central laboratory for Compound (I) receptor occupancy and α4β7 expression and other exploratory PD assessments.

### Sample per Subject

Blood (4 mL) samples for PK analysis will be obtained in K₂EDTA tubes prior to dosing and at the time points delineated in the study schedule. All samples (except those collected post-dose on Day 0 and Day 84) should be obtained prior to the daily dosing of study drug (i.e. C_{trough}).

Pharmacodynamic assessments will be measured from 2 mL blood samples collected in heparinized blood collection tubes from subjects at selected sites according to the study schedule).

The actual collection time of each sample must be recorded in the source data, collection tube, and on the electronic CRF (eCRF). The allowed time deviation window for blood sample collection is ±20 minutes for the samples up to 4 hours post-dose on Days 0 and 84.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

The present invention may be embodied in other specific forms without departing from its structures, methods, or other essential characteristics as broadly described herein and claimed hereinafter. The described embodiments are to be considered in all respects only as illustrative, and not restrictive. The scope of the invention is, therefore, indicated by the appended claims, rather than by the foregoing description. All changes that come within the meaning and range of equivalency of the claims are to be embraced within their scope.

1. A method of treating an inflammatory bowel disease (IBD) in a subject in need thereof, the method comprising orally administering to the subject a compound of formula (I): 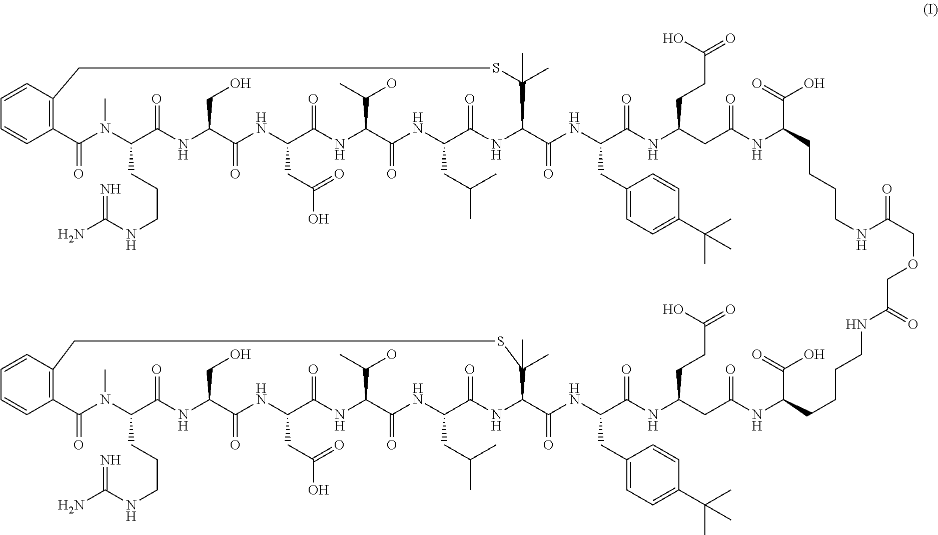 [SMILES: CC(C)C[C@@H]1NC(=O)[C@H](C(C)O)NC(=O)[C@H](CC(=O)O)NC(=O)[C@H](CO)NC(=O)[C@H](CCCNC(=N)N)N(C)C(=O)c2ccccc2CSC(C)(C)[C@@H](C(=O)N[C@@H](Cc2ccc(C(C)(C)C)cc2)C(=O)N[C@@H](CCC(=O)O)CC(=O)N[C@H](CCCCNC(=O)COCC(=O)NCCCC[C@@H](NC(=O)C[C@H](CCC(=O)O)NC(=O)[C@H](Cc2ccc(C(C)(C)C)cc2)NC(=O)[C@H]2NC(=O)[C@H](CC(C)C)NC(=O)[C@H](C(C)O)NC(=O)[C@H](CC(=O)O)NC(=O)[C@H](CO)NC(=O)[C@H](CCCNC(=N)N)N(C)C(=O)c3ccccc3CSC2(C)C)C(=O)O)C(=O)O)NC1=O]
or a pharmaceutically acceptable salt thereof, or a pharmaceutical composition comprising a compound of formula (I) or a pharmaceutically acceptable salt thereof, wherein the subject has a plasma concentration of the compound of from about 0.1 ng/mL to about 4 ng/mL following administration of the compound or pharmaceutically acceptable salt thereof, and wherein the administering compound of formula (I) or pharmaceutically acceptable salt thereof to the subject achieves a clinical remission rate of at least about 6% higher relative to a clinical remission rate from placebo.

2. The method of claim 1, wherein the plasma concentration is between about 0.2 and about 0.7 ng/mL.

3. The method of claim 1, wherein the compound or pharmaceutically acceptable salt thereof is administered at a dose from about 50 mg to about 1000 mg per day as divided doses or as a once daily dose.

4. The method of claim 1, wherein the compound or pharmaceutically acceptable salt thereof is administered to the subject at a dose of about 50, 62.5, 75, 87.5, 100.0, 112.5, 125.0, 137.5, 150.0, 162.5, 175, 187.5, 200.0, 212.5, 225.0, 237.5, 250.0, 262.5, 275, 287.5, 300.0, 312.5, 325.0, 337.5, 350.0, 362.5, 375, 387.5, 400.0, 412.5, 425.0, 437.5, 450.0, 462.5, 475, 487.5, 500.0, 512.5, 525, 537.5, 550, 562.5, 575, 587.5, 600, 612.5, 625, 637.5, 650, 662.5, 675, 687.5, 700, 712.5, 725, 737.5, 750, 762.5, 775, 787.5,800, 812.5, 825, 837.5, 850, 862.5, 875, 887.5, 900, 912.5, 925, 937.5, 950, 962.5, 975, 987.5 or 1000 mg.

5. The method of claim 1, wherein the compound or pharmaceutically acceptable salt thereof is administered at a dose of 150 mg or 450 mg once or twice per day.

6. The method of claim 1, wherein the administering of the compound of formula (I) or pharmaceutically acceptable salt thereof to the subject achieves the clinical remission rate of from about 13% to about 40% higher relative to the clinical remission rate from placebo.

7. The method of claim 1, wherein the administering of the compound of formula (I) or pharmaceutically acceptable sat thereof to the subject achieves the clinical remission rate of at least 40% higher relative to the clinical remission rate from placebo.

8. The method of claim 1, wherein the administering of the compound of formula (I) or pharmaceutically acceptable salt thereof to the subject achieves a histologic remission rate of at least about 20% higher relative to the histologic remission rate from placebo.

9. The method of claim 1, wherein the administering of the compound of formula (I) or pharmaceutically acceptable salt thereof to the subject achieves a histologic remission rate of at least about 20% to about 37% higher relative to the histologic remission rate from placebo.

10. A method of optimizing or improving therapeutic efficacy for treating IBD in a subject in need thereof, the method comprising orally administering a compound of formula (I): 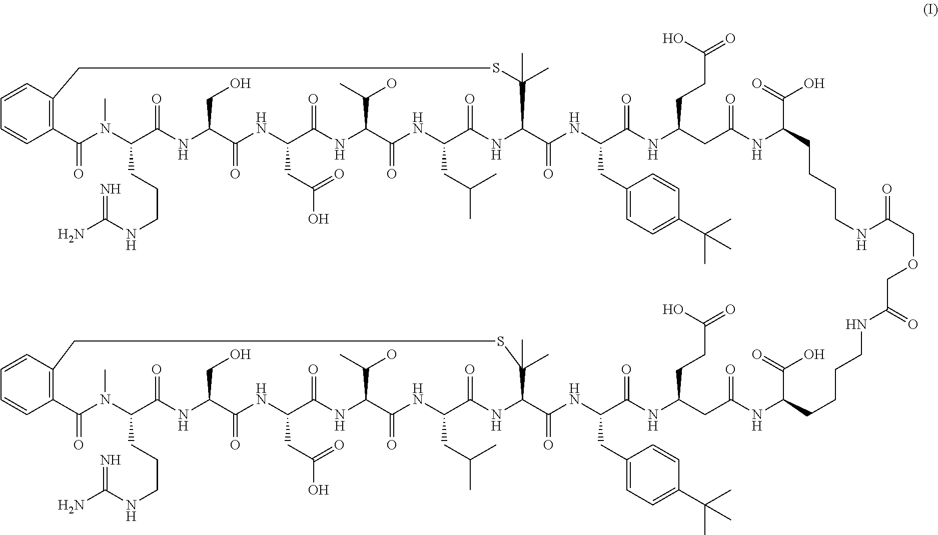 [SMILES: CC(C)C[C@@H]1NC(=O)[C@H](C(C)O)NC(=O)[C@H](CC(=O)O)NC(=O)[C@H](CO)NC(=O)[C@H](CCCNC(=N)N)N(C)C(=O)c2ccccc2CSC(C)(C)[C@@H](C(=O)N[C@@H](Cc2ccc(C(C)(C)C)cc2)C(=O)N[C@@H](CCC(=O)O)CC(=O)N[C@H](CCCCNC(=O)COCC(=O)NCCCC[C@@H](NC(=O)C[C@H](CCC(=O)O)NC(=O)[C@H](Cc2ccc(C(C)(C)C)cc2)NC(=O)[C@H]2NC(=O)[C@H](CC(C)C)NC(=O)[C@H](C(C)O)NC(=O)[C@H](CC(=O)O)NC(=O)[C@H](CO)NC(=O)[C@H](CCCNC(=N)N)N(C)C(=O)c3ccccc3CSC2(C)C)C(=O)O)C(=O)O)NC1=O]
or a pharmaceutically acceptable salt thereof, or a pharmaceutical composition comprising a compound of formula (I) or a pharmaceutically acceptable salt thereof, to the subject, wherein the plasma concentration of the compound of formula (I) or pharmaceutically acceptable salt thereof in the subject is maintained at a predetermined concentration.

11. The method of claim 10, wherein the predetermined plasma concentration is between about 0.1 and about 0.85 ng/mL.

12. The method of claim 10, wherein the predetermined plasma concentration is between about 0.3 and about 0.7 ng/mL.

13. The method of claim 10, wherein the plasma concentration is maintained by determining the plasma concentration of the compound of formula (I) or pharmaceutically acceptable salt thereof in the subject; and adjusting administering the compound of formula (I) or a pharmaceutically acceptable salt thereof, or a pharmaceutical composition comprising a compound of formula (I) or a pharmaceutically acceptable salt thereof, to achieve a desired therapeutic effect.

14. The method of claim 13, wherein the adjusting comprises changing the dosage regimen of the compound or pharmaceutically acceptable salt thereof administered to the subject.

15. The method of claim 1, wherein the compound of formula (I) or pharmaceutically acceptable salt thereof is administered twice daily.

16. The method of claim 1, wherein the subject is a human patient.

17. The method of claim 1, wherein the IBD is ulcerative colitis.

18. The method of claim 1, wherein the subject has moderate to severe ulcerative colitis.

19. The method of claim 1, wherein the IBD is Crohn's disease.
